(19) United States
(12) Patent Application Publication
(10) Pub. No.: US 2010/0029491 A1
(43) Pub. Date: Feb. 4, 2010

(54) METHODS AND COMPOSITIONS FOR DIAGNOSTIC USE FOR TUMOR TREATMENT

(76) Inventors: Maike Schmidt, San Francisco, CA (US); Anil D. Bagri, San Carlos, CA (US)

Correspondence Address: GENENTECH, INC., 1 DNA WAY, SOUTH SAN FRANCISCO, CA, 94080, US

(21) Appl. No.: 12/499,692

(22) Filed: Jul. 8, 2009

Related U.S. Application Data
(60) Provisional application No. 61/080,173, filed on Jul. 11, 2008.

(51) Int. Cl. C40B 30/00; C12Q 1/68

(52) U.S. Cl. 506/7; 435/6

(57) ABSTRACT

Diagnostic markers for tumors, and their use in the diagnosis and treatment of tumors are provided.

### RELATED APPLICATIONS

This application claims benefit from U.S. Provisional Application No. 61/080,173, filed Jul. 11, 2008.

### FIELD OF THE INVENTION

The invention relates to diagnostic methods and compositions useful in the treatment of cancer.

### BACKGROUND OF THE INVENTION

Cancer is one of the most deadly threats to human health. In the U.S. alone, cancer affects nearly 1.3 million new patients each year, and is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

Depending on the cancer type, patients typically have several treatment options available to them including chemotherapy, radiation and antibody-based drugs. Diagnostic methods useful for predicting clinical outcome from the different treatment regimens would greatly benefit clinical management of these patients. Several studies have explored the correlation of gene expression with the identification of specific cancer types, e.g., by mutation-specific assays, microarray analysis, qPCR, etc. Such methods may be useful for the identification and classification of cancer presented by a patient. However, much less is known about the predictive or prognostic value of gene expression with clinical outcome.

Thus, there is a need for objective, reproducible methods for the optimal treatment regimen for each patient.

### SUMMARY OF THE INVENTION

The methods of the present invention can be utilized in a variety of settings, including, for example, in selecting patient for a treatment course, in prediction of likelihood of success when treating an individual patient with a particular treatment regimen, in assessing disease progression, in monitoring treatment efficacy, in determining prognosis for individual patients and in assessing predisposition of an individual to benefit from a particular anti-cancer therapy in addition to anti-angiogenic therapy.

Methods of identifying a patient who may benefit from anti-cancer therapy in addition to VEGF antagonist, methods of predicting responsiveness of a patient to anti-angiogenic therapy in addition to VEGF antagonist, and methods of determining likelihood of clinical benefit to a patient from anti-cancer therapy in addition to VEGF antagonist are provided herein. For example, methods comprise determining the ratio between expression level of a gene and expression level of VEGF-A in a sample obtained from a patient, wherein a change in the ratio between the expression level of said gene and the expression level of VEGF-A in the sample as compared to the ratio between the expression level of said gene and the expression level of VEGF-A in a reference sample indicates that the patient may benefit from anti-cancer therapy in addition to VEGF antagonist, that the patient is likely to be responsive to anti-angiogenic therapy in addition to VEGF antagonist and/or increased likelihood of clinical benefit to the patient from anti-cancer therapy in addition to VEGF antagonist.

In certain embodiments of the methods, the change in the ratio is an increase. In another embodiment the change in the ratio is a decrease. In certain embodiments, the gene is an angiogenic factor. In certain embodiments, the expression level is mRNA expression level. In one embodiment, the mRNA expression is in tumor cells. In certain embodiments, the change in the ratio between the mRNA expression level of said angiogenic factor and the mRNA expression level of VEGF-A is an increase. In one embodiment, the angiogenic factor is VEGF-C. In another embodiment, the angiogenic factor is VEGF-D. In yet another embodiment, the angiogenic factor is bFGF. In yet another embodiment, the angiogenic factor is VEGFR3.

In certain embodiments, the angiogenic factor is VEGF-C and the methods further comprise determining the ratio between expression level of VEGF-D and expression level of VEGF-A in the sample, wherein the ratio between expression level of VEGF-D and expression level of VEGF-A in the sample is increased as compared to the ratio between expression level of VEGF-D and expression level of VEGF-A in the reference sample. In one embodiment, the increased ratio in the sample compared to the reference sample indicates that the patient may benefit from anti-cancer therapy in addition to VEGF antagonist, the patient is likely to be responsive to anti-angiogenic therapy in addition to VEGF antagonist and/or increased likelihood of clinical benefit to the patient from anti-cancer therapy in addition to VEGF antagonist. In certain embodiments, the expression level is mRNA expression level. In certain embodiments, the expression level is protein expression level.

In certain embodiments, the angiogenic factor is VEGF-C and the methods further comprise determining the ratio between expression level of bFGF and expression level of VEGF-A in the sample, wherein the ratio between expression level of bFGF and expression level of VEGF-A in the sample is increased as compared to the ratio between expression level of bFGF and expression level of VEGF-A in the reference sample. In one embodiment, the increased ratio in the sample compared to the reference sample indicates that the patient may benefit from anti-cancer therapy in addition to VEGF antagonist, the patient is likely to be responsive to anti-angiogenic therapy in addition to VEGF antagonist and/or increased likelihood of clinical benefit to the patient from anti-cancer therapy in addition to VEGF antagonist. In certain embodiments, the expression level is mRNA expression level. In certain embodiments, the expression level is protein expression level.

In certain embodiments, the angiogenic factor is VEGF-D and the methods further comprise determining the ratio between expression level of VEGF-C and expression level of VEGF-A in the sample, wherein the ratio between expression level of VEGF-C and expression level of VEGF-A in the sample is increased as compared to the ratio between expression level of VEGF-C and expression level of VEGF-A in the reference sample. In one embodiment, the increased ratio in the sample compared to the reference sample indicates that the patient may benefit from anti-cancer therapy in addition to VEGF antagonist, the patient is likely to be responsive to anti-angiogenic therapy in addition to VEGF antagonist and/or increased likelihood of clinical benefit to the patient from anti-cancer therapy in addition to VEGF antagonist. In certain embodiments, the expression level is mRNA expression level. In certain embodiments, the expression level is protein expression level.

In certain embodiments, the angiogenic factor is VEGF-D and the methods further comprise determining the ratio between expression level of bFGF and expression level of VEGF-A in the sample, wherein the ratio between expression level of bFGF and expression level of VEGF-A in the sample is increased as compared to the ratio between expression level of bFGF and expression level of VEGF-A in the reference sample. In one embodiment, the increased ratio in the sample compared to the reference sample indicates that the patient may benefit from anti-cancer therapy in addition to VEGF antagonist, the patient is likely to be responsive to anti-angiogenic therapy in addition to VEGF antagonist and/or increased likelihood of clinical benefit to the patient from anti-cancer therapy in addition to VEGF antagonist. In certain embodiments, the expression level is mRNA expression level. In certain embodiments, the expression level is protein expression level.

In certain embodiments, the angiogenic factor is bFGF and the methods further comprise determining the ratio between expression level of VEGF-C and expression level of VEGF-A in the sample, wherein the ratio between expression level of VEGF-C and expression level of VEGF-A in the sample is increased as compared to the ratio between expression level of VEGF-C and expression level of VEGF-A in the reference sample. In one embodiment, the increased ratio in the sample compared to the reference sample indicates that the patient may benefit from anti-cancer therapy in addition to VEGF antagonist, the patient is likely to be responsive to anti-angiogenic therapy in addition to VEGF antagonist and/or increased likelihood of clinical benefit to the patient from anti-cancer therapy in addition to VEGF antagonist. In certain embodiments, the expression level is mRNA expression level. In certain embodiments, the expression level is protein expression level.

In certain embodiments, the angiogenic factor is bFGF and the methods further comprise determining the ratio between expression level of VEGF-D and expression level of VEGF-A in the sample, wherein the ratio between expression level of VEGF-D and expression level of VEGF-A in the sample is increased as compared to the ratio between expression level of VEGF-D and expression level of VEGF-A in the reference sample. In one embodiment, the increased ratio in the sample compared to the reference sample indicates that the patient may benefit from anti-cancer therapy in addition to VEGF antagonist, the patient is likely to be responsive to anti-angiogenic therapy in addition to VEGF antagonist and/or increased likelihood of clinical benefit to the patient from anti-cancer therapy in addition to VEGF antagonist. In certain embodiments, the expression level is mRNA expression level. In certain embodiments, the expression level is protein expression level.

In one aspect, methods are provided which include identifying a patient who may benefit from anti-cancer therapy in addition to VEGF antagonist, comprising determining the ratio between expression level of a gene and expression level of VEGF-A in a sample obtained from a patient, wherein the ratio of 0.1 or greater between the expression level of said gene and expression level of VEGF-A indicates that the patient may benefit from anti-cancer therapy in addition to VEGF antagonist. In one embodiment, the ratio is 0.25 or greater between the expression level of said gene and expression level of VEGF-A. In another embodiment, the ratio is 0.5 or greater between the expression level of said gene and expression level of VEGF-A. In yet another embodiment, the ratio is 1.0 or greater between the expression level of said gene and expression level of VEGF-A. In certain embodiments, said gene is VEGF-C. In certain embodiments, said gene is VEGF-D. In certain embodiments, said gene is bFGF. In certain embodiments, said gene is VEGFR3.

In one aspect, methods are provided which include predicting responsiveness of a patient to anti-angiogenic therapy comprising determining the ratio between expression level of a gene and expression level of VEGF-A in a sample obtained from a patient, wherein the ratio of 0.1 or greater between the expression level of said gene and expression level of VEGF-A indicates that the patient is likely to be responsive to anti-angiogenic therapy in addition to VEGF antagonist. In one embodiment, the ratio is 0.25 or greater between the expression level of said gene and expression level of VEGF-A. In another embodiment, the ratio is 0.5 or greater between the expression level of said gene and expression level of VEGF-A. In yet another embodiment, the ratio is 1.0 or greater between the expression level of said gene and expression level of VEGF-A. In certain embodiments, said gene is VEGF-C. In certain embodiments, said gene is VEGF-D. In certain embodiments, said gene is bFGF. In certain embodiments, said gene is VEGFR3.

In one aspect, methods are provided which include determining likelihood of clinical benefit from anti-cancer therapy in addition to VEGF antagonist comprising determining a ratio between expression level of a gene and expression level of VEGF-A in a sample obtained from a patient, wherein the ratio of 0.1 or greater between the expression level of said gene and expression level of VEGF-A indicates increased likelihood of clinical benefit to the patient from anti-cancer therapy in addition to VEGF antagonist. In one embodiment, the ratio is 0.25 or greater between the expression level of said gene and expression level of VEGF-A. In another embodiment, the ratio is 0.5 or greater between the expression level of said gene and expression level of VEGF-A. In yet another embodiment, the ratio is 1.0 or greater between the expression level of said gene and expression level of VEGF-A. In certain embodiments, said gene is VEGF-C. In certain embodiments, said gene is VEGF-D. In certain embodiments, said gene is bFGF. In certain embodiments, said gene is VEGFR3.

In one aspect, methods of determining a ratio between expression level of a gene and expression level of VEGF-A in a sample are provided, where the methods comprise:
(a) determining relative expression of said gene in the sample;
(b) determining relative expression of said gene in a reference sample;
(c) determining normalized relative expression of said gene in the sample comprising dividing the relative expression of said gene in the sample by the relative expression of said gene in the reference sample;
(d) determining relative expression of VEGF-A in the sample;
(e) determining relative expression of VEGF-A in a reference sample;
(f) determining normalized relative expression of VEGF-A in the sample comprising dividing the relative expression of VEGF-A in the sample by the relative expression of VEGF-A in the reference sample; and
(g) determining the ratio between the expression level of said gene and the expression level of VEGF-A by dividing the normalized expression of said gene by the normalized expression of VEGF-A.

In certain embodiments, the gene is an angiogenic factor or its receptor. In some embodiments, angiogenic factors include, but not limited to, angiogenic factors and their receptors as defined herein under Definitions.

In certain embodiments, the expression level of the gene or the angiogenic factor is mRNA expression level. In certain embodiments, the expression level of the gene or the angiogenic factor is protein expression level.

In certain embodiments, the mRNA expression level of the gene or the angiogenic factor is measured using qRT-PCR or qPCR. In another embodiment, the mRNA expression level is measured using microarray. In another embodiment, the mRNA expression level is measured using ISH (in situ hybridization). In certain embodiments, the protein expression level of the gene or the angiogenic factor is measured using IHC assay.

In certain embodiments, the change in the ratio between the mRNA expression level of the angiogenic factor and the mRNA expression levels of VEGF-A is an increase. In one embodiment, the angiogenic factor is VEGF-C. In another embodiment, the angiogenic factor is VEGF-D. In yet another embodiment, the angiogenic factor is bFGF. In yet another embodiment, the angiogenic factor is VEGFR3.

In one embodiment, the ratio between the mRNA expression level of a gene and the mRNA expression level of VEGF-A in a sample from a patient is at least 0.5 and said gene is VEGF-C. In another embodiment, the ratio between the mRNA expression level of a gene and the mRNA expression level of VEGF-A in a sample from a patient is at least 1 and said gene is VEGF-C. In one embodiment, the ratio between the mRNA expression level of a gene and the mRNA expression level of VEGF-A in a sample from a patient is at least 0.1 and said gene is VEGF-D. In one embodiment, the ratio between the mRNA expression level of a gene and the mRNA expression level of VEGF-A in a sample from a patient is at least 0.25 and said gene is VEGF-D. In another embodiment, the ratio between the mRNA expression level of a gene and the mRNA expression level of VEGF-A is at least 0.5 and said gene is VEGF-D. In one embodiment, the ratio between the mRNA expression level of a gene and the mRNA expression level of VEGF-A in a sample from a patient is at least 0.5 and said gene is bFGF. In another embodiment, the ratio between the mRNA expression level of a gene and the mRNA expression level of VEGF-A in a sample from a patient is at least 1 and said gene is bFGF. In yet another embodiment, the ratio between the mRNA expression level of a gene and the mRNA expression level of VEGF-A in a sample from a patient is at least 2 and said gene is bFGF.

In certain embodiments, the VEGF antagonist therapy comprises administration of anti-VEGF antibody. In certain embodiments, the VEGF antagonist is an anti-VEGF antibody, a VEGF-Trap (e.g., VEGF receptor-Fc fusion) or an anti-VEGF receptor antibody. In certain embodiments, the VEGF antagonist is an anti-VEGF antibody. In certain embodiments, the anti-VEGF antibody is a human or humanized anti-VEGF antibody. In one embodiment, the anti-VEGF antibody is bevacizumab.

In certain embodiments, the methods of the present invention further comprise administering to the patient an effective amount of anti-cancer therapeutic agent in addition to VEGF antagonist. In certain embodiments, anti-cancer therapeutic agent is VEGF-C antagonist. In certain embodiments, the VEGF-C antagonist is an anti-VEGF-C antibody. In certain embodiments, an effective amount of anti-VEGF-C antibody in addition to VEGF antagonist is administered to the patient. In certain embodiments, an effective amount of anti-VEGF-C antibody in addition to anti-VEGF antibody is administered to the patient. In certain embodiments, anti-VEGF-C antibody and anti-VEGF antibody are administered simultaneously to the patient.

In certain embodiments, methods for treating cancer in a patient are provided. For example, the method comprises determining that the ratio between expression level of a gene and expression level of VEGF-A in a sample obtained from the patient has changed as compared to the ratio between the expression level of said gene and the expression level of VEGF-A in a reference sample, and administering an effective amount of an anti-cancer therapy other than a VEGF antagonist to said patient, whereby the cancer is treated. In certain embodiments, the cancer is resistant tumor. In certain embodiments, the patient is relapsed from or refractory to anti-cancer therapy comprising VEGF antagonist. In certain embodiments, the patient is relapsed from or refractory to anti-cancer therapy comprising anti-VEGF antibody. In certain embodiments, the patient is relapsed from or refractory to anti-cancer therapy comprising bevacizumab.

In certain embodiments, methods for treating a cell proliferative disorder in a patient are provided. For example, the methods comprise determining that the ratio between expression level of a gene and expression level of VEGF-A in a sample obtained from the patient has changed as compared to the ratio between the expression level of said gene and the expression level of VEGF-A in a reference sample, and administering an effective amount of an anti-cancer therapy other than a VEGF antagonist to said patient, whereby the cell proliferative disorder is treated.

In certain embodiments, methods for inhibiting angiogenesis in a patient are provided. The methods comprise determining that the ratio between expression level of a gene and expression level of VEGF-A in a sample obtained from the patient has changed as compared to the ratio between the expression level of said gene and the expression level of VEGF-A in a reference sample, and administering an effective amount of an anti-cancer therapy other than a VEGF antagonist to said patient.

In certain embodiments, methods for inhibiting lymphangiogenesis in a patient are provided. The methods comprise determining that the ratio between expression level of a gene and expression level of VEGF-A in a sample obtained from the patient has changed as compared to the ratio between the expression level of said gene and the expression level of VEGF-A in a reference sample, and administering an effective amount of an anti-cancer therapy other than a VEGF antagonist to said patient.

In certain embodiments, the anti-cancer agent or anti-angiogenic agent, including but not limited to anti-VEGF antibody and anti-VEGF-C antibody, is administered to the patient according to the instructions provided on the label or package insert.

In one embodiment, the change in the ratio is an increase. In another embodiment the change in the ratio is a decrease. In certain embodiments, the gene is an angiogenic factor. In certain embodiments, the expression level is mRNA expression level. In one embodiment, the mRNA expression is in tumor cells. In certain embodiments, the change in the ratio between the mRNA expression level of said angiogenic factor and the mRNA expression level of VEGF-A is an increase. In one embodiment, the angiogenic factor is VEGF-C. In another embodiment, the angiogenic factor is VEGF-D. In yet another embodiment, the angiogenic factor is bFGF. In yet another embodiment, the angiogenic factor is VEGFR3.

In certain embodiments, the sample obtained from the patient is tissue, blood, serum or any combination thereof. In certain embodiments, the sample obtained from the patient is a tissue sample.

In certain embodiments, the methods of the present invention can further comprise determining immunohistochemistry (IHC) score comprising performing IHC assay for the gene, wherein the IHC score is at least 1+. In one embodiment, the IHC score is at least 2+. In yet another embodiment, the IHC score is 3+.

In certain embodiments, the methods of the present invention can further comprise determining whether the sample comprises a tumor cell that expresses VEGFR3, wherein presence of VEGFR3 expression indicates that the patient may benefit from anti-cancer therapy in addition to VEGF antagonist, that the patient is likely to be responsive to anti-angiogenic therapy in addition to VEGF antagonist and/or increased likelihood of clinical benefit to the patient from anti-cancer therapy in addition to VEGF antagonist.

In one embodiment, the VEGFR3 expression is mRNA expression. In another embodiment, the presence of VEGFR3 mRNA expression is determined using qRT-PCR or qPCR. In yet another embodiment, the presence of VEGFR3 protein expression is determined using IHC assay.

In certain embodiments, the methods of the present invention can further comprise measuring expression level of VEGFR3, wherein the expression level of VEGFR3 in the sample is increased as compared to the reference sample. In one embodiment, the expression level of VEGFR3 is mRNA expression level. In another embodiment, the increased mRNA expression level of VEGFR3 is in tumor cells.

In certain embodiments, the methods of the present invention can further comprise determining immunohistochemistry (IHC) score comprising performing IHC assay for the gene and determining whether the sample comprises a tumor cell that expresses VEGFR3, wherein the IHC score of at least 1+ and presence of VEGFR3 expression indicate that the patient may benefit from anti-cancer therapy in addition to VEGF antagonist, that the patient is likely to be responsive from anti-angiogenic therapy and/or increased likelihood of clinical benefit to the patient from anti-cancer therapy in addition to VEGF antagonist. In one embodiment, the IHC score is at least 2+. In yet another embodiment, the IHC score is 3+. In one embodiment, the expression level of VEGFR3 is mRNA expression level. In another embodiment, the increased mRNA expression level of VEGFR3 is in tumor cells.

In certain embodiments, the methods of the present invention can further comprise determining whether the sample comprises a tumor cell that expresses VEGF-D, wherein presence of VEGF-D expression indicates that the patient may benefit from anti-cancer therapy in addition to VEGF antagonist, that the patient is likely to be responsive to anti-angiogenic therapy in addition to VEGF antagonist and/or increased likelihood of clinical benefit to the patient from anti-cancer therapy in addition to VEGF antagonist. In one embodiment, the VEGF-D expression is mRNA expression. In another embodiment, the presence of VEGF-D mRNA expression is determined using qRT-PCR or qPCR. In another embodiment, the VEGF-D expression is protein expression. In another embodiment, the presence of VEGF-D protein expression is determined using IHC assay.

In one aspect, the invention provides a kit comprising an array comprising polynucleotides capable of specifically hybridizing to one or more genes and to VEGF-A, wherein the kit further comprises instructions for using said array to determine ratios between the expression levels of one of more said genes and VEGF-A to predict responsiveness of a patient to anti-angiogenic therapy or anti-cancer therapy, wherein a change in the ratio between the expression level of at least one of said genes and the expression level of VEGF-A in the sample as compared to the ratio between the expression level of the same gene and expression level of VEGF-A in a reference sample indicates that the patient may benefit from anti-angiogenic therapy or anti-cancer therapy in addition to VEGF antagonist.

In certain embodiments, the ratio of 0.1 or greater between the expression level of at least one of said genes and the expression level of VEGF-A in the sample indicates that the patient may benefit from anti-angiogenic therapy or anti-cancer therapy in addition to VEGF antagonist. In certain embodiments, the ratio of 0.25 or greater between the expression level of at least one of said genes and the expression level of VEGF-A indicates that the patient may benefit from anti-angiogenic therapy or anti-cancer therapy in addition to VEGF antagonist. In certain embodiments, the ratio of 0.5 or greater between the expression level of at least one of said genes and the expression level of VEGF-A indicates that the patient may benefit from anti-angiogenic therapy or anti-cancer therapy in addition to VEGF antagonist. In certain embodiments, the ratio of 1 or greater between the expression level of at least one of said genes and the expression level of VEGF-A indicates that the patient may benefit from anti-angiogenic therapy or anti-cancer therapy in addition to VEGF antagonist. In certain embodiments, the ratio of 2 or greater between the expression level of at least one of said genes and the expression level of VEGF-A indicates that the patient may benefit from anti-angiogenic therapy or anti-cancer therapy in addition to VEGF antagonist.

In one aspect, the invention provides a set of compounds capable of detecting expression levels of one or more genes and expression level of VEGF-A to determine ratios between expression levels of one or more genes and expression level of VEGF-A in a sample obtained from the patient, wherein a change in the ratio between the expression level of at least one of said genes and the expression level of VEGF-A in the sample as compared to the ratio between the expression level of the same gene and the expression level of VEGF-A in a reference sample indicates that the patient may benefit from anti-cancer therapy in addition to VEGF antagonist. In certain embodiments, the compounds are polynucleotides. In certain embodiments, the compounds are proteins. In one embodiment, the proteins are antibodies.

In one aspect, the invention provides a set of compounds capable of detecting expression levels of one or more genes and expression level of VEGF-A to determine ratios between expression levels of one or more genes and expression level of VEGF-A in a sample obtained from the patient, wherein the ratio of 0.1 or greater between the expression level of at least one of said genes and the expression level of VEGF-A in the sample indicates that the patient may benefit from anti-angiogenesis or anti-cancer therapy in addition to VEGF antagonist. In certain embodiments, the ratio is 0.25 or greater. In certain embodiments, the ratio is 0.5 or greater. In certain embodiments, the ratio is 1 or greater. In certain embodiments, the ratio is 2 or greater. In certain embodiments, the compounds are polynucleotides. In certain embodiments, the compounds are proteins. In one embodiment, the proteins are antibodies.

In certain embodiments, the patient is diagnosed with cancer. In certain embodiments, the cancer is carcinoma, lymphoma, blastoma, sarcoma, and leukemia. More particular examples of such cancers include, but not limited to, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors) or Meigs' syndrome. In certain embodiment, the cancer is colorectal cancer, non-small cell lung cancer, breast cancer, glioblastoma or renal cancer.

In certain embodiments, the methods of the invention can be performed with anti-angiogenic therapies comprising administration of an anti-angiogenic agents such as, but not limited to, antibodies to or antagonists of VEGF-A or the VEGF-A receptor (e.g., KDR receptor or Flt-1 receptor) and antibodies to or antagonists of VEGF-C. In certain embodiments, methods of the invention further comprise administering one or more anti-angiogenic agents and/or anti-cancer agents in addition to VEGF antagonist. In certain embodiments the VEGF antagonist is an anti-VEGF antibody. In one embodiment, the anti-VEGF antibody is bevacizumab. In certain embodiments, the anti-angiogenic agent administered in addition to VEGF antagonist is an antibody to or antagonist of VEGF-C. In one embodiment, the anti-angiogenic agent is anti-VEGF-C antibody. Additional list of anti-angiogenic agents can be found herein under Definitions and Angiogenic Inhibitors.

Any embodiment described above or any combination thereof applies to any and all methods of the inventions described herein.

### BRIEF DESCRIPTION OF THE DRAWINGS

FIGS. 1A and 1B: Graphs showing relative VEGF-A mRNA expression for tumor xenograft models. FIG. 1B shows the same data as FIG. 1A, but with normalization to two housekeeping genes, and increased number of tumor samples per model.

FIGS. 2A and 2B: Graphs showing relative VEGF-C mRNA expression for tumor xenograft models. FIG. 2B shows the same data as FIG. 2A, but with normalization to two housekeeping genes, and increased number of tumor samples per model.

FIGS. 3A and 3B: Graphs showing relative VEGF-D mRNA expression for tumor xenograft models. FIG. 3B shows the same data as FIG. 3A, but with normalization to two housekeeping genes, and increased number of tumor samples per model.

FIGS. 4A and 4B: Graphs showing relative VEGFR3 mRNA expression for tumor xenograft models. FIG. 4B shows the same data as FIG. 4A, but with normalization to two housekeeping genes, and increased number of tumor samples per model.

FIG. 5: Graph showing relative bFGF mRNA expression for tumor xenograft models.

FIGS. 6A and 6B: Graphs showing a correlation between increase in efficacy and VEGF-C to VEGF-A relative mRNA expression ratio. FIG. 6B shows the same data as FIG. 6A, but with normalization to two housekeeping genes, alternative Δ % TGD calculation, and increased number of tumor samples per model.

FIGS. 7A and 7B: Graph showing a correlation between increase in efficacy and VEGF-D to VEGF-A relative mRNA expression ratio. FIG. 7B shows the same data as FIG. 7A, but with normalization to two housekeeping genes, alternative Δ % TGD calculation, and increased number of tumor samples per model.

FIG. 8: Graph showing a correlation between increase in efficacy and bFGF to VEGF-A relative mRNA expression ratio.

FIG. 9: Graph showing a correlation between efficacy and VEGF-C/VEGF-A and VEGF-D/VEGF-A relative mRNA expression ratios.

FIG. 10: Graph showing a correlation between efficacy and VEGF-C/VEGF-A and bFGF/VEGF-A relative mRNA expression ratios.

FIG. 11: Graph showing a correlation between efficacy and VEGF-D/VEGF-A and bFGF/VEGF-A relative mRNA expression ratios.

FIG. 12: Illustration of VEGF-C IHC score for human ovarian adenocarcinoma.

FIG. 13: VEGF-C IHC stained sections of H460 and A549 tumors.

FIGS. 14A and 14B: Graphs showing a correlation between increase in efficacy and VEGF-C IHC score. FIG. 14B shows the same data as FIG. 14A, but with alternative Δ % TGD calculation.

### DETAILED DESCRIPTION OF THE INVENTION

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. All references cited herein, including patent applications and publications, are incorporated by reference in their entirety.

### DEFINITIONS

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

An “individual,” “subject,” or “patient” is a vertebrate. In certain embodiments, the vertebrate is a mammal. Mammals include, but are not limited to, farm animals (such as cows), sport animals, pets (such as cats, dogs, and horses), primates, mice and rats. In certain embodiments, a mammal is a human.

The term “sample,” or “test sample” as used herein, refers to a composition that is obtained or derived from a subject of interest that contains a cellular and/or other molecular entity that is to be characterized and/or identified, for example based on physical, biochemical, chemical and/or physiological characteristics. In one embodiment, the definition encompasses blood and other liquid samples of biological origin and tissue samples such as a biopsy specimen or tissue cultures or cells derived therefrom. The source of the tissue sample may be solid tissue as from a fresh, frozen and/or preserved organ or tissue sample or biopsy or aspirate; blood or any blood constituents; bodily fluids; and cells from any time in gestation or development of the subject or plasma.

In another embodiment, the definition includes biological samples that have been manipulated in any way after their procurement, such as by treatment with reagents, solubilization, or enrichment for certain components, such as proteins or polynucleotides, or embedding in a semi-solid or solid matrix for sectioning purposes. For the purposes herein a “section” of a tissue sample is meant a single part or piece of a tissue sample, e.g. a thin slice of tissue or cells cut from a tissue sample.

Samples include, but not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, urine, cerebro-spinal fluid, saliva, sputum, tears, perspiration, mucus, tumor lysates, and tissue culture medium, as well as tissue extracts such as homogenized tissue, tumor tissue, and cellular extracts.

In one embodiment, the sample is a clinical sample. In another embodiment, the sample is used in a diagnostic assay. In some embodiments, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For instance, samples of lung cancer lesions may be obtained by resection, bronchoscopy, fine needle aspiration, bronchial brushings, or from sputum, pleural fluid or blood.

In one embodiment, a sample is obtained from a subject or patient prior to anti-angiogenic therapy. In another embodiment, a sample is obtained from a subject or patient prior to VEGF antagonist therapy. In yet another embodiment, a sample is obtained from a subject or patient prior to anti-VEGF antibody therapy. In certain embodiments, a sample is obtained after cancer has metastasized.

A “reference sample,” as used herein, refers to any sample, standard, or level that is used for comparison purposes. In one embodiment, a reference sample is obtained from a healthy and/or non-diseased part of the body of the same subject or patient. In another embodiment, a reference sample is obtained from an untreated tissue and/or cell of the body of the same subject or patient.

In certain embodiments, a reference sample is a single sample or combined multiple samples from the same subject or patient that are obtained at one or more different time points than when the test sample is obtained. For example, a reference sample is obtained at an earlier time point from the same subject or patient than when the test sample is obtained. Such reference sample may be useful if the reference sample is obtained during initial diagnosis of cancer and the test sample is later obtained when the cancer becomes metastatic.

In one embodiment, a reference sample is obtained from a healthy and/or non-diseased part of the body of an individual who is not the subject or patient. In another embodiment, a reference sample is obtained from an untreated tissue and/or cell part of the body of an individual who is not the subject or patient.

In certain embodiments, a reference sample includes all types of biological samples as defined above under the term “sample” that is obtained from one or more individuals who is not the subject or patient. In certain embodiments, a reference sample is obtained from one or more individuals with cancer who is not the subject or patient.

In certain embodiments, a reference sample is a combined multiple samples from one or more healthy individuals who are not the subject or patient. In certain embodiments, a reference sample is a combined multiple samples from one or more individuals with cancer who are not the subject or patient. In certain embodiments, a reference sample is pooled RNA samples from normal tissues from one or more individuals who are not the subject or patient. In certain embodiments, a reference sample is pooled RNA samples from tumor tissues from one or more individuals with cancer who are not the subject or patient.

Expression levels/amount of a gene or biomarker can be determined qualitatively and/or quantitatively based on any suitable criterion known in the art, including but not limited to mRNA, cDNA, proteins, protein fragments and/or gene copy number. In certain embodiments, expression/amount of a gene or biomarker in a first sample is increased as compared to expression/amount in a second sample. In certain embodiments, expression/amount of a gene or biomarker in a first sample is decreased as compared to expression/amount in a second sample. In certain embodiments, the second sample is reference sample.

In certain embodiments, the term “increase” refers to an overall increase of 5%, 10%, 20%, 25%, 30%, 40%, 50%, 60%, 70%, 80%, 85%, 90%, 95%, 96%, 97%, 98%, 99% or greater, in the level of protein or nucleic acid, detected by standard art known methods such as those described herein, as compared to a reference sample. In certain embodiments, the term increase refers to the increase in expression level/amount of a gene or biomarker in the sample wherein the increase is at least about 1.1×, 1.2×, 1.3×, 1.4×, 1.5×, 1.6×, 1.7×, 1.75×, 1.8×, 1.9×, 2×, 3×, 4×, 5×, 6×, 7×, 8×, 9×, 10×, 25×, 50×, 75×, or 100× the expression level/amount of the respective gene or biomarker in the reference sample.

In certain embodiments, the term “decrease” herein refers to an overall reduction of 5%, 10%, 20%, 25%, 30%, 40%, 50%, 60%, 70%, 80%, 85%, 90%, 95%, 96%, 97%, 98%, 99% or greater, in the level of protein or nucleic acid, detected by standard art known methods such as those described herein, as compared to a reference sample. In certain embodiments, the term decrease refers to the decrease in expression level/amount of a gene or biomarker in the sample wherein the decrease is at least about 0.9×, 0.8×, 0.7×, 0.6×, 0.5×, 0.4×, 0.3×, 0.2×, 0.1×, 0.05×, or 0.01× the expression level/amount of the respective gene or biomarker in the reference sample.

Additional disclosures for determining expression level/amount of a gene are described herein under Methods of the Invention.

A “ratio,” as used herein, refers to a quantity that denotes the proportional amount or magnitude of one quantity relative to another. Ratios are generally unitless when they relate quantities of the same dimension. Fractions and percentages are both specific applications of ratios. Fractions relate the part (the numerator) to the whole (the denominator) while percentages indicate parts per 100.

Additional disclosures for determining ratios between expression level/amount of a gene and expression level of VEGF-A in the sample and in the reference sample or changes in the ratios are described herein under Methods of the Invention.

“Detection” includes any means of detecting, including direct and indirect detection.

In certain embodiments, by “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

The word “label” when used herein refers to a compound or composition which is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable.

A “small molecule” is defined herein to have a molecular weight below about 500 Daltons.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs.

“Oligonucleotide,” as used herein, generally refers to short, generally single-stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

In certain embodiments, polynucleotides are capable of specifically hybridizing to a gene under various stringency conditions. “Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

Stringent conditions or high stringency conditions may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

Moderately stringent conditions may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

An “isolated” nucleic acid molecule is a nucleic acid molecule that is identified and separated from at least one contaminant nucleic acid molecule with which it is ordinarily associated in the natural source of the polypeptide nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the polypeptide where, for example, the nucleic acid molecule is in a chromosomal location different from that of natural cells.

A “primer” is generally a short single stranded polynucleotide, generally with a free 3′-OH group, that binds to a target potentially present in a sample of interest by hybridizing with a target sequence, and thereafter promotes polymerization of a polynucleotide complementary to the target.

The term “housekeeping gene” refers to a group of genes that codes for proteins whose activities are essential for the maintenance of cell function. These genes are typically similarly expressed in all cell types. In certain embodiments, one or more housekeeping genes are used to determine the relative expression level of a gene.

The term “biomarker” as used herein refers generally to a molecule, including a gene, protein, carbohydrate structure, or glycolipid, the expression of which in or on a mammalian tissue or cell can be detected by standard methods (or methods disclosed herein) and is predictive, diagnostic and/or prognostic for a mammalian cell's or tissue's sensitivity to treatment regimes based on inhibition of angiogenesis e.g. an anti-angiogenic agent such as a VEGF-specific inhibitor. In certain embodiments, the biomarker is a gene. In certain embodiments, the expression of such a biomarker is determined to be higher or lower than that observed for a reference sample. Expression of such biomarkers can be determined using a high-throughput multiplexed immunoassay such as those commercially available from Rules Based Medicine, Inc. or Meso Scale Discovery. Expression of the biomarkers may also be determined using, e.g., PCR or FACS assay, an immunohistochemical assay or a gene chip-based assay.

The term “array” or “microarray,” as used herein refers to an ordered arrangement of hybridizable array elements, preferably polynucleotide probes (e.g., oligonucleotides), on a substrate. The substrate can be a solid substrate, such as a glass slide, or a semi-solid substrate, such as nitrocellulose membrane. The nucleotide sequences can be DNA, RNA, or any permutations thereof.

A “target gene,” “target biomarker,” “target sequence,” “target nucleic acid” or “target protein,” as used herein, is a polynucleotide or protein of interest, the detection of which is desired. Generally, a “template,” as used herein, is a polynucleotide that contains the target nucleotide sequence. In some instances, the terms “target sequence,” “template DNA,” “template polynucleotide,” “target nucleic acid,” “target polynucleotide,” and variations thereof, are used interchangeably.

“Amplification,” as used herein, generally refers to the process of producing multiple copies of a desired sequence. “Multiple copies” mean at least 2 copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

A “native sequence” polypeptide comprises a polypeptide having the same amino acid sequence as a polypeptide derived from nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

An “isolated” polypeptide or “isolated” antibody is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In certain embodiments, the polypeptide will be purified (1) to greater than 95% by weight of polypeptide as determined by the Lowry method, or more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or silver stain. Isolated polypeptide includes the polypeptide in situ within recombinant cells since at least one component of the polypeptide's natural environment will not be present. Ordinarily, however, isolated polypeptide will be prepared by at least one purification step.

A polypeptide “variant” means a biologically active polypeptide having at least about 80% amino acid sequence identity with the native sequence polypeptide. Such variants include, for instance, polypeptides wherein one or more amino acid residues are added, or deleted, at the N- or C-terminus of the polypeptide. Ordinarily, a variant will have at least about 80% amino acid sequence identity, more preferably at least about 90% amino acid sequence identity, and even more preferably at least about 95% amino acid sequence identity with the native sequence polypeptide.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

Unless indicated otherwise, the expression “multivalent antibody” denotes an antibody comprising three or more antigen binding sites. In certain embodiment, the multivalent antibody is engineered to have the three or more antigen binding sites and is generally not a native sequence IgM or IgA antibody.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma &Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

“Framework” or “FR” residues are those variable domain residues other than the HVR residues as herein defined.

An “affinity matured” antibody is one with one or more alterations in one or more HVRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies may be produced using certain procedures known in the art. For example, Marks et al. Bio/Technology 10:779-783 (1992) describes affinity maturation by VH and VL domain shuffling. Random mutagenesis of HVR and/or framework residues is described by, for example, Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See also, e.g., Shields et al. J. Biol. Chem. 9(2):6591-6604 (2001).

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

“Antibody-dependent cell-mediated cytotoxicity” or “ADCC” refers to a form of cytotoxicity in which secreted Ig bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

The term “Fc region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, a composition comprising an antibody having an Fc region according to this invention can comprise an antibody with K447, with all K447 removed, or a mixture of antibodies with and without the K447 residue.

A “blocking” antibody or an “antagonist” antibody is one which inhibits or reduces biological activity of the antigen it binds. For example, a VEGF-specific antagonist antibody binds VEGF and inhibits the ability of VEGF to induce vascular endothelial cell proliferation or vascular permeability. Certain blocking antibodies or antagonist antibodies substantially or completely inhibit the biological activity of the antigen.

The term “VEGF” or “VEGF-A” as used herein refers to the 165-amino acid human vascular endothelial cell growth factor and related 121-, 189-, and 206-amino acid human vascular endothelial cell growth factors, as described by Leung et al. (1989) Science 246:1306, and Houck et al. (1991) Mol. Endocrin, 5:1806, together with the naturally occurring allelic and processed forms thereof. The term “VEGF” also refers to VEGFs from non-human species such as mouse, rat or primate. Sometimes the VEGF from a specific species are indicated by terms such as hVEGF for human VEGF, mVEGF for murine VEGF, and etc. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

“VEGF biological activity” includes binding to any VEGF receptor or any VEGF signaling activity such as regulation of both normal and abnormal angiogenesis and vasculogenesis (Ferrara and Davis-Smyth (1997) Endocrine Rev. 18:4-25; Ferrara (1999) J. Mol. Med. 77:527-543); promoting embryonic vasculogenesis and angiogenesis (Carmeliet et al. (1996) Nature 380:435-439; Ferrara et al. (1996) Nature 380:439-442); and modulating the cyclical blood vessel proliferation in the female reproductive tract and for bone growth and cartilage formation (Ferrara et al. (1998) Nature Med. 4:336-340; Gerber et al. (1999) Nature Med. 5:623-628). In addition to being an angiogenic factor in angiogenesis and vasculogenesis, VEGF, as a pleiotropic growth factor, exhibits multiple biological effects in other physiological processes, such as endothelial cell survival, vessel permeability and vasodilation, monocyte chemotaxis and calcium influx (Ferrara and Davis-Smyth (1997), supra and Cebe-Suarez et al. Cell. Mol. Life. Sci. 63:601-615 (2006)). Moreover, recent studies have reported mitogenic effects of VEGF on a few non-endothelial cell types, such as retinal pigment epithelial cells, pancreatic duct cells, and Schwann cells. Guerrin et al. (1995) J. Cell Physiol. 164:385-394; Oberg-Welsh et al. (1997) Mol. Cell. Endocrinol. 126:125-132; Sondell et al. (1999) J. Neurosci. 19:5731-5740.

A “VEGF antagonist” or “VEGF-specific antagonist” refers to a molecule capable of neutralizing, blocking, inhibiting, abrogating, reducing or interfering with VEGF activities including, but not limited to, its binding to one or more VEGF receptors. VEGF antagonists include, without limitation, anti-VEGF antibodies and antigen-binding fragments thereof, receptor molecules and derivatives which bind specifically to VEGF thereby sequestering its binding to one or more receptors, anti-VEGF receptor antibodies and VEGF receptor antagonists such as small molecule inhibitors of the VEGFR tyrosine kinases. The term “VEGF antagonist,” as used herein, specifically includes molecules, including antibodies, antibody fragments, other binding polypeptides, peptides, and non-peptide small molecules, that bind to VEGF and are capable of neutralizing, blocking, inhibiting, abrogating, reducing or interfering with VEGF activities. Thus, the term “VEGF activities” specifically includes VEGF mediated biological activities of VEGF. In certain embodiments, the VEGF antagonist reduces or inhibits, by at least 10%, 20%, 30%, 40%, 50%, 60%, 70%, 80%, 90% or more, the expression level or biological activity of VEGF.

An “anti-VEGF antibody” is an antibody that binds to VEGF with sufficient affinity and specificity. In certain embodiments, the antibody selected will normally have a sufficiently binding affinity for VEGF, for example, the antibody may bind hVEGF with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example.

In certain embodiment, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PlGF, PDGF or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody (see Presta et al. (1997) Cancer Res. 57:4593-4599), including but not limited to the antibody known as bevacizumab (BV; AVASTIN®).

The anti-VEGF antibody “Bevacizumab (BV),” also known as “rhuMAb VEGF” or “AVASTIN®,” comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005, the entire disclosure of which is expressly incorporated herein by reference.

The term “B20 series polypeptide” as used herein refers to a polypeptide, including an antibody that binds to VEGF. B20 series polypeptides includes, but not limited to, antibodies derived from a sequence of the B20 antibody or a B20-derived antibody described in US Publication No. 20060280747, US Publication No. 20070141065 and/or US Publication No. 20070020267, the content of these patent applications are expressly incorporated herein by reference. In one embodiment, B20 series polypeptide is B20-4.1 as described in US Publication No. 20060280747, US Publication No. 20070141065 and/or US Publication No. 20070020267. In another embodiment, B20 series polypeptide is B20-4.1.1 described in U.S. patent application Ser. No. 12/315,221, the entire disclosure of which is expressly incorporated herein by reference.

The term “G6 series polypeptide” as used herein refers to a polypeptide, including an antibody that binds to VEGF. G6 series polypeptides includes, but not limited to, antibodies derived from a sequence of the G6 antibody or a G6-derived antibody described in US Publication No. 20060280747, US Publication No. 20070141065 and/or US Publication No. 20070020267. G6 series polypeptides, as described in US Publication No. 20060280747, US Publication No. 20070141065 and/or US Publication No. 20070020267 include, but not limited to, G6-8, G6-23 and G6-31.

For additional antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004). In certain embodiments, other antibodies include those that bind to a functional epitope on human VEGF comprising of residues F17, M18, D19, Y21, Y25, Q89, I91, K101, E103, and C104 or, alternatively, comprising residues F17, Y21, Q22, Y25, D63, 183 and Q89.

Other anti-VEGF antibodies, anti-Nrp1 antibodies and anti-Nrp2 antibodies are also known, and described, for example, in Liang et al., J Mol Biol 366, 815-829 (2007) and Liang et al., J Biol Chem 281, 951-961 (2006), PCT publication number WO2007/056470 and PCT Application No. PCT/US2007/069179, the contents of these patent applications are expressly incorporated herein by reference.

VEGF-C, a member of the VEGF family, is known to bind at least two cell surface receptor families, the tyrosine kinase VEGF receptors and the neuropilin (Nrp) receptors. Of the three VEGF receptors, VEGF-C can bind VEGFR2 (KDR receptor) and VEGFR3 (Flt-4 receptor) leading to receptor dimerization (Shinkai et al., J Biol Chem 273, 31283-31288 (1998)), kinase activation and autophosphorylation (Heldin, Cell 80, 213-223 (1995); Waltenberger et al., J. Biol Chem 269, 26988-26995 (1994)). The phosphorylated receptor induces the activation of multiple substrates leading to angiogenesis and lymphangiogenesis (Ferrara et al., Nat Med 9, 669-676 (2003)). VEGF-C is one of the best studied mediators of lymphatic development. Overexpression of VEGF-C in tumor cells was shown to promote tumor-associated lymphangiogenesis, resulting in enhanced metastasis to regional lymph nodes (Karpanen et al., Faseb J 20, 1462-1472 (2001); Mandriota et al., EMBO J 20, 672-682 (2001); Skobe et al., Nat Med 7, 192-198 (2001); Stacker et al., Nat Rev Cancer 2, 573-583 (2002); Stacker et al., Faseb J 16, 922-934 (2002)). VEGF-C expression has also been correlated with tumor-associated lymphangiogenesis and lymph node metastasis for a number of human cancers (reviewed in Achen et al., 2006, supra). In addition, blockade of VEGF-C-mediated signaling has been shown to suppress tumor lymphangiogenesis and lymph node metastases in mice (Chen et al., Cancer Res 65, 9004-9011 (2005); He et al., J. Natl Cancer Inst 94, 8190825 (2002); Krishnan et al., Cancer Res 63, 713-722 (2003); Lin et al., Cancer Res 65, 6901-6909 (2005)).

The term “VEGF-C” refers to the full-length polypeptide and/or the active fragments of the full-length polypeptide. In one embodiment, active fragments include any portions of the full-length amino acid sequence which have less than the full 419 amino acids of the full-length amino acid sequence as shown in SEQ ID NO:3 of U.S. Pat. No. 6,451,764, the entire disclosure of which is expressly incorporated herein by reference. Such active fragments contain VEGF-C biological activity and include, but not limited to, mature VEGF-C. In one embodiment, the full-length VEGF-C polypeptide is proteolytically processed produce a mature form of VEGF-C polypeptide, also referred to as mature VEGF-C. Such processing includes cleavage of a signal peptide and cleavage of an amino-terminal peptide and cleavage of a carboxyl-terminal peptide to produce a fully-processed mature form. Experimental evidence demonstrates that the full-length VEGF-C, partially-processed forms of VEGF-C and fully processed mature forms of VEGF-C are able to bind VEGFR3 (Flt-4 receptor). However, high affinity binding to VEGFR2 occurs only with the fully processed mature forms of VEGF-C.

The term “VEGF-C antagonist” is used herein to refer to a molecule capable of neutralizing, blocking, inhibiting, abrogating, reducing or interfering with the ability of VEGF-C to modulate angiogenesis, lymphatic endothelial cell (EC) migration, proliferation or adult lymphangiogenesis, especially tumoral lymphangiogenesis and tumor metastasis. VEGF-C antagonists include, without limitation, anti-VEGF-C antibodies and antigen-binding fragments thereof, receptor molecules and derivatives which bind specifically to VEGF-C thereby sequestering its binding to one or more receptors, anti-VEGF-C receptor antibodies and VEGF-C receptor antagonists such as small molecule inhibitors of the VEGFR2 and VEGFR3. Anti-VEGF-C antibodies are described, for example, in Attorney Docket PR4291, the entire content of the patent application is expressly incorporated herein by reference. The term “VEGF-C antagonist,” as used herein, specifically includes molecules, including antibodies, antibody fragments, other binding polypeptides, peptides, and non-peptide small molecules, that bind to VEGF-C and are capable of neutralizing, blocking, inhibiting, abrogating, reducing or interfering with VEGF-C activities. Thus, the term “VEGF-C activities” specifically includes VEGF-C mediated biological activities (as hereinabove defined) of VEGF-C.

VEGF-D, a member of the VEGF family, is recognized by VEGF receptors VEGFR2 (KDR receptor) and VEGFR3 (Flt-4 receptor) (see Marconcini et al. PNAS 96, 9671-9676 (1999); Baldwin et al. J Biol Chem 276, 19166-19171 (2001)). VEGF-D is most closely related to VEGF-C in the VEGF-family. VEGF-D is initially synthesized as a precursor protein containing N- and C-terminal propeptides. The N- and C-terminal propeptides are proteolytically cleaved to generate a mature VEGF-D (Stacker et al. J Biol Chem 274, 32127-32136 (1999)).

The term “VEGF-D” refers to the full-length polypeptide and/or the active fragments of the full-length polypeptide. In one embodiment, active fragments include any portions of the full-length amino acid sequence which have less than the full 354 amino acids of the full-length amino acid sequence as shown in SEQ ID NO:1 of U.S. Pat. No. 6,828,426, the entire disclosure of which is expressly incorporated herein by reference. Such active fragments contain VEGF-D biological activity and include, but not limited to, mature VEGF-D. In one embodiment, the full-length VEGF-D polypeptide is proteolytically processed produce a mature form of VEGF-D polypeptide, also referred to as mature VEGF-D.

Additional disclosures relating to VEGF-D are described in, for example, Achen et al. PNAS 95, 548-553 (1998), US Publication No. 2005/0112665, U.S. Pat. No. 6,235,713, and U.S. Pat. No. 6,689,580, the entire disclosure of which are expressly incorporated herein by reference.

VEGFR3 is endothelial specific receptor tyrosine kinase, regulated by members of the vascular endothelial growth factor family. VEGF-C and VEGF-D are both ligands for VEGFR3. (see Marconcini et al. PNAS 96, 9671-9676 (1999); Baldwin et al. J Biol Chem 276, 19166-19171 (2001); Stacker et al. J Biol Chem 274, 32127-32136 (1999); Achen et al. PNAS 95, 548-553 (1998)).

The term “VEGFR3” or “Flt4” refers to the full-length polypeptide and/or fragments of the full-length polypeptide. In one embodiment, fragments include any portions of the full-length amino acid sequence which have less than 1298 amino acids of the full-length amino acid sequence as shown in SEQ ID NO:2 of U.S. Pat. No. 6,824,777, the entire disclosure of which is expressly incorporated herein by reference. In one embodiment, fragments include any portions of the full-length amino acid sequence which have less than 1363 amino acids of the full-length amino acid sequence as shown in SEQ ID NO:4 of U.S. Pat. No. 6,824,777.

Additional disclosures relating to VEGFR3 are described in, for example, U.S. Pat. No. 6,824,777, and U.S. Pat. No. 7,034,105, the entire disclosure of which are expressly incorporated herein by reference.

The term “bFGF”, also known as “FGF2”, “FGF-β” or “basic fibroblast growth factor”, is a member of the fibroblast growth factor family. bFGF stimulates the proliferation of all cells of mesodermal origin including smooth muscle cells, neuroblasts, and endothelial cells. bFGF stimulates neuron differentiation, survival, and regeneration. In vitro functions suggest that bFGF modulates angiogenesis, wound healing and tissue repair, and neuronal function in vivo. bFGF, a heparin-binding growth factor, is capable of inducing functionally significant angiogenesis in models of myocardial and limb ischemia. Zheng, et al., Am. J. Physiol. Heart Circ. Physiol., 280: H909-17 (2001), Laham, et al., J. Am. Coll. Cardiol., 36: 2132-39 (2000), Laham, et al., Curr. Interv. Cardiol. Rep., 1: 228 (1999), Unger, et al., Am. J. Cardiol., 85: 1414-19 (2000), Kawasuji, et al., Ann. Thorac. Surg., 69: 1155 (2000), Rajanayagam, et al., J. Am. Coll. Cardiol., 35: 519 (2000), Kornowski, et al., Circulation, 101: 545-48 (2000), Ohara, et al., Gene Ther., 8: 837 (2001), Lazarous, et al., J. Am. Coll. Cardiol., 36: 1239 (2000), Rakue, et al., Japan Circ. J., 62: 933-39 (1998), Baffour, et al., J. Vasc. Surg., 16: 181 (1992).

As used herein, “treatment” (and variations such as “treat” or “treating”) refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, methods and compositions of the invention are used to delay development of a disease or disorder or to slow the progression of a disease or disorder.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

In the case of pre-cancerous, benign, early or late-stage tumors, the therapeutically effective amount of the angiogenic inhibitor may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit or delay, to some extent, tumor growth or tumor progression; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

To “reduce or inhibit” is to decrease or reduce an activity, function, and/or amount as compared to a reference. In certain embodiments, by “reduce or inhibit” is meant the ability to cause an overall decrease of 20% or greater. In another embodiment, by “reduce or inhibit” is meant the ability to cause an overall decrease of 50% or greater. In yet another embodiment, by “reduce or inhibit” is meant the ability to cause an overall decrease of 75%, 85%, 90%, 95%, or greater. Reduce or inhibit can refer to the symptoms of the disorder being treated, the presence or size of metastases, the size of the primary tumor, or the size or number of the blood vessels in angiogenic disorders.

A “disorder” is any condition that would benefit from treatment. For example, mammals who suffer from or need prophylaxis against abnormal angiogenesis (excessive, inappropriate or uncontrolled angiogenesis) or vascular permeability. This includes chronic and acute disorders or diseases including those pathological conditions which predispose the mammal to the disorder in question. Non-limiting examples of disorders to be treated herein include malignant and benign tumors; non-leukemias and lymphoid malignancies; and, in particular, tumor (cancer) metastasis.

The terms “cell proliferative disorder” and “proliferative disorder” refer to disorders that are associated with some degree of abnormal cell proliferation. In one embodiment, the cell proliferative disorder is cancer.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, ovarian cancer, mesothelioma, and multiple myeloma. In some embodiments, the cancer is selected from the group consisting of small cell lung cancer, gliblastoma, neuroblastomas, melanoma, breast carcinoma, gastric cancer, colorectal cancer (CRC), and hepatocellular carcinoma. Yet, in some embodiments, the cancer is selected from the group consisting of non-small cell lung cancer, colorectal cancer, renal cancer, glioblastoma and breast carcinoma, including metastatic forms of those cancers.

The term “resistant tumor” refers to cancer, cancerous cells, or a tumor that does not respond completely, or loses or shows a reduced response over the course of cancer therapy to a cancer therapy comprising at least a VEGF antagonist. In certain embodiments, resistant tumor is a tumor that is resistant to anti-VEGF antibody therapy. In one embodiment, the anti-VEGF antibody is bevacizumab. In certain embodiments, resistant tumor is a tumor that is unlikely to respond to a cancer therapy comprising at least a VEGF antagonist. In certain embodiments, responsiveness to a cancer therapy is the responsiveness of a patient to a cancer therapy as defined herein.

“Refractory” refers to the resistance or non-responsiveness of a disease or condition to a treatment (e.g., the number of neoplastic plasma cells increases even though treatment if given). Unless otherwise indicated, the term “refractory” refers a resistance or non-responsiveness to any previous treatment including, but not limited to, VEGF antagonist and chemotherapy treatments. In certain embodiments, VEGF antagonist is an anti-VEGF antibody.

“Relapsed” refers to the regression of the patient's illness back to its former diseased state, especially the return of symptoms following an apparent recovery or partial recovery. Unless otherwise indicted, relapsed state refers to the process of returning to or the return to illness before the previous treatment including, but not limited to, VEGF antagonist and chemotherapy treatments. In certain embodiments, VEGF antagonist is an anti-VEGF antibody.

The term “anti-cancer therapy” or “cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenic agents, apoptotic agents, anti-tubulin agents, and other agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva™), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

An “angiogenic factor or agent” is a growth factor or its receptor which is involved in stimulating the development of blood vessels, e.g., promote angiogenesis, endothelial cell growth, stability of blood vessels, and/or vasculogenesis, etc. For example, angiogenic factors, include, but are not limited to, e.g., VEGF and members of the VEGF family and their receptors (VEGF-B, VEGF-C, VEGF-D, VEGFR1, VEGFR2 and VEGFR3), PlGF, PDGF family, fibroblast growth factor family (FGFs), TIE ligands (Angiopoietins, ANGPT1, ANGPT2), TIE1, TIE2, ephrins, Bv8, Delta-like ligand 4 (DLL4), EGF-like-domain, multiple 7 (EGFL7), Del-1, fibroblast growth factors: acidic (aFGF) and basic (bFGF), FGF4, FGF9, BMP9, BMP10, Follistatin, Granulocyte colony-stimulating factor (G-CSF), GM-CSF, Hepatocyte growth factor (HGF)/scatter factor (SF), Interleukin-8 (IL-8), CXCL12, Leptin, Midkine, neuropilins, NRP1, NRP2, Placental growth factor, Platelet-derived endothelial cell growth factor (PD-ECGF), Platelet-derived growth factor, especially PDGF-BB, PDGFR-alpha, or PDGFR-beta, Pleiotrophin (PTN), Progranulin, Proliferin, Transforming growth factor-alpha (TGF-alpha), Transforming growth factor-beta (TGF-beta), Tumor necrosis factor-alpha (TNF-alpha), Alk1, CXCR4, Notch1, Notch4, Sema3A, Sema3C, Sema3F, Robo4, ESM1, Perlecan, etc. It would also include factors that accelerate wound healing, such as growth hormone, insulin-like growth factor-I (IGF-I), VIGF, epidermal growth factor (EGF), CTGF and members of its family, and TGF-alpha and TGF-beta. See, e.g., Klagsbrun and D'Amore (1991) Annu. Rev. Physiol. 53:217-39; Streit and Detmar (2003) Oncogene 22:3172-3179; Ferrara & Alitalo (1999) Nature Medicine 5(12):1359-1364; Tonini et al. (2003) Oncogene 22:6549-6556 (e.g., Table 1 listing known angiogenic factors); and, Sato (2003) Int. J. Clin. Oncol. 8:200-206.

An “anti-angiogenic agent” or “angiogenic inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenic agent includes those agents that bind and block the angiogenic activity of the angiogenic factor or its receptor. For example, an anti-angiogenic agent is an antibody or other antagonist to an angiogenic agent as defined above, e.g., antibodies to VEGF-A or to the VEGF-A receptor (e.g., KDR receptor or Flt-1 receptor), antibodies to VEGF-C, anti-PDGFR inhibitors, small molecules that block VEGF receptor signaling (e.g., PTK787/ZK2284, SU6668, SUTENT®/SU11248 (sunitinib malate), AMG706, or those described in, e.g., international patent application WO 2004/113304). Anti-angiogenic agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12): 1359-1364; Tonini et al. (2003) Oncogene 22:6549-6556 (e.g., Table 2 listing known antiangiogenic factors); and, Sato (2003) Int. J. Clin. Oncol. 8:200-206 (e.g., Table 1 listing anti-angiogenic agents used in clinical trials). Additional exemplary and non-limiting list of angiogenic inhibitors are provided herein under “Angiogenic Inhibitors.”

The term “anti-angiogenic therapy” refers to a therapy useful for inhibiting angiogenesis which comprises the administration of an anti-angiogenic agent.

The term “cytotoxic agent” as used herein refers to a substance that inhibits or prevents a cellular function and/or causes cell death or destruction. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu), chemotherapeutic agents (e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “toxin” is any substance capable of having a detrimental effect on the growth or proliferation of a cell.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhôme-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylomithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifamib (R11577); orafenib, ABT510; Bcl-2 inhibitor such as oblimersen sodium (GENASLENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON• toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above; as well as combinations of two or more of the above.

A “growth inhibitory agent” when used herein refers to a compound or composition which inhibits growth of a cell either in vitro or in vivo. In one embodiment, growth inhibitory agent is growth inhibitory antibody that prevents or reduces proliferation of a cell expressing an antigen to which the antibody binds. In another embodiment, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

The term “pharmaceutical formulation” refers to a preparation which is in such form as to permit the biological activity of the active ingredient to be effective, and which contains no additional components which are unacceptably toxic to a subject to which the formulation would be administered. Such formulations may be sterile.

A “sterile” formulation is aseptic or free from all living microorganisms and their spores.

Administration “in combination with” one or more further therapeutic agents includes simultaneous (concurrent) and consecutive administration in any order.

The term “simultaneously” or “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A “liposome” is a small vesicle composed of various types of lipids, phospholipids and/or surfactant which is useful for delivery of a drug (such as a VEGF-C polypeptide or antibody thereto) to a mammal. The components of the liposome are commonly arranged in a bilayer formation, similar to the lipid arrangement of biological membranes.

The term “diagnosis” is used herein to refer to the identification of a molecular or pathological state, disease or condition, such as the identification of cancer or to refer to identification of a cancer patient who may benefit from a particular treatment regimen.

The term “prognosis” is used herein to refer to the prediction of the likelihood of clinical benefit from anti-cancer therapy.

The term “prediction” is used herein to refer to the likelihood that a patient will respond either favorably or unfavorably to a particular anti-cancer therapy. In one embodiment, the prediction relates to the extent of those responses. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc., or whether long-term survival of the patient, following a therapeutic regimen is likely.

Responsiveness of a patient can be assessed using any endpoint indicating a benefit to the patient, including, without limitation, (1) inhibition, to some extent, of disease progression, including slowing down and complete arrest; (2) reduction in lesion size; (3) inhibition (i.e., reduction, slowing down or complete stopping) of disease cell infiltration into adjacent peripheral organs and/or tissues; (4) inhibition (i.e. reduction, slowing down or complete stopping) of disease spread; (5) relief, to some extent, of one or more symptoms associated with the disorder; (6) increase in the length of disease-free presentation following treatment; and/or (8) decreased mortality at a given point of time following treatment.

The term “benefit” is used in the broadest sense and refers to any desirable effect and specifically includes clinical benefit as defined herein.

Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

### Angiogenic Inhibitors

Anti-angiogenic agents include, but are not limited to, the following agents: VEGF inhibitors such as a VEGF-specific antagonist, VEGF-C inhibitors such as a VEGF-C specific antagonist, EGF inhibitor, EGFR inhibitors, Erbitux® (cetuximab, ImClone Systems, Inc., Branchburg, N.J.), Vectibix® (panitumumab, Amgen, Thousand Oaks, Calif.), TIE2 inhibitors, IGF1R inhibitors, COX-II (cyclooxygenase II) inhibitors, MMP-2 (matrix-metalloprotienase 2) inhibitors, and MMP-9 (matrix-metalloprotienase 9) inhibitors, CP-547,632 (Pfizer Inc., NY, USA), Axitinib (Pfizer Inc.; AG-013736), ZD-6474 (AstraZeneca), AEE788 (Novartis), AZD-2171), VEGF Trap (Regeneron/Aventis), Vatalanib (also known as PTK-787, ZK-222584: Novartis & Schering A G), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof. Other angiogenesis inhibitors include thrombospondin1, thrombospondin2, collagen IV and collagen XVIII. VEGF inhibitors are disclosed in U.S. Pat. Nos. 6,534,524 and 6,235,764, both of which are incorporated in their entirety for all purposes.

A VEGF-specific antagonist refers to a molecule capable of binding to VEGF, reducing VEGF expression levels, or neutralizing, blocking, inhibiting, abrogating, reducing, or interfering with VEGF biological activities, including VEGF binding to one or more VEGF receptors and VEGF mediated angiogenesis and endothelial cell survival or proliferation. Included as VEGF-specific antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF₁₂₁-gelonin (Peregrine). VEGF-specific antagonists also include antagonist variants of VEGF polypeptides, antisense nucleobase oligomers directed to VEGF, small RNA molecules directed to VEGF, RNA aptamers, peptibodies, and ribozymes against VEGF.

The two best characterized VEGF receptors are VEGFR1 (also known as Flt-1) and VEGFR2 (also known as KDR and FLK-1 for the murine homolog). The specificity of each receptor for each VEGF family member varies but VEGF-A binds to both Flt-1 and KDR. The full length Flt-1 receptor includes an extracellular domain that has seven Ig domains, a transmembrane domain, and an intracellular domain with tyrosine kinase activity. The extracellular domain is involved in the binding of VEGF and the intracellular domain is involved in signal transduction.

VEGF receptor molecules, or fragments thereof, that specifically bind to VEGF can be used as VEGF inhibitors that bind to and sequester the VEGF protein, thereby preventing it from signaling. In certain embodiments, the VEGF receptor molecule, or VEGF binding fragment thereof, is a soluble form, such as sFlt-1. A soluble form of the receptor exerts an inhibitory effect on the biological activity of the VEGF protein by binding to VEGF, thereby preventing it from binding to its natural receptors present on the surface of target cells. Also included are VEGF receptor fusion proteins, examples of which are described below.

A chimeric VEGF receptor protein is a receptor molecule having amino acid sequences derived from at least two different proteins, at least one of which is a VEGF receptor protein (e.g., the Flt-1 or KDR receptor), that is capable of binding to and inhibiting the biological activity of VEGF. In certain embodiments, the chimeric VEGF receptor proteins of the present invention consist of amino acid sequences derived from only two different VEGF receptor molecules; however, amino acid sequences comprising one, two, three, four, five, six, or all seven Ig-like domains from the extracellular ligand-binding region of the Flt-1 and/or KDR receptor can be linked to amino acid sequences from other unrelated proteins, for example, immunoglobulin sequences. Other amino acid sequences to which Ig-like domains are combined will be readily apparent to those of ordinary skill in the art. Examples of chimeric VEGF receptor proteins include, but not limited to, soluble Flt-1/Fc, KDR/Fc, or Flt-1/KDR/Fc (also known as VEGF Trap). (See for example PCT Application Publication No. WO97/44453).

A soluble VEGF receptor protein or chimeric VEGF receptor proteins includes VEGF receptor proteins which are not fixed to the surface of cells via a transmembrane domain. As such, soluble forms of the VEGF receptor, including chimeric receptor proteins, while capable of binding to and inactivating VEGF, do not comprise a transmembrane domain and thus generally do not become associated with the cell membrane of cells in which the molecule is expressed.

Additional VEGF inhibitors are described in, for example in WO 99/24440, PCT International Application PCT/IB99/00797, in WO 95/21613, WO 99/61422, U.S. Pat. No. 6,534,524, U.S. Pat. No. 5,834,504, WO 98/50356, U.S. Pat. No. 5,883,113, U.S. Pat. No. 5,886,020, U.S. Pat. No. 5,792,783, U.S. Pat. No. 6,653,308, WO 99/10349, WO 97/32856, WO 97/22596, WO 98/54093, WO 98/02438, WO 99/16755, and WO 98/02437, all of which are herein incorporated by reference in their entirety.

### METHODS OF THE INVENTION

The present invention is based partly on the identification of specific genes or biomarkers that correlate with identifying patients who may benefit from anti-cancer therapy in addition to VEGF antagonist for treating cancer. Thus, the disclosed methods provide convenient, efficient, and potentially cost-effective means to obtain data and information useful in assessing appropriate or effective therapies for treating cancer patients. For example, a cancer patient could have a biopsy performed to obtain a tissue or cell sample, and the sample could be examined by various in vitro assays to determine whether the ratio between the expression level of a particular gene, e.g., VEGF-C and VEGF-A in a sample has changed as compared to the ratio between the expression level of the gene and VEGF-A in a reference sample. If a change, e.g., increase, in ratio is detected the patient will probably benefit from anti-cancer therapy in addition to VEGF antagonist.

In certain embodiments, expression levels/amount of a gene can be determined based on any suitable criterion known in the art, including but not limited to mRNA, cDNA, proteins, protein fragments and/or gene copy number.

In certain embodiments, expression of various genes in a sample can be analyzed by a number of methodologies, many of which are known in the art and understood by the skilled artisan, including but not limited to, immunohistochemical and/or Western blot analysis, immunoprecipitation, molecular binding assays, ELISA, ELIFA, fluorescence activated cell sorting (FACS) and the like, quantitative blood based assays (as for example Serum ELISA) (to examine, for example, levels of protein expression), biochemical enzymatic activity assays, in situ hybridization, Northern analysis and/or PCR analysis of mRNAs, as well as any one of the wide variety of assays that can be performed by gene and/or tissue array analysis. Typical protocols for evaluating the status of genes and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (MSD) may also be used.

In certain embodiments, expression/amount of a gene in a sample is increased as compared to expression/amount in a reference sample if the expression level/amount of the gene in the sample is greater than the expression level/amount of the gene in the reference sample. Similarly, expression/amount of a gene in a sample is decreased as compared to expression/amount in a reference sample if the expression level/amount of the gene in the ample is less than the expression level/amount of the gene in the reference sample.

In certain embodiments, the change in the ratio between the expression level of a gene and the expression level of VEGF-A in the sample as compared to the same ratio in the reference sample is an increase. In certain embodiments, the ratio between expression level of a gene and VEGF-A in a sample is increased as compared to the ratio of the expression levels of the gene and VEGF-A in a reference sample if the increase in the expression level of the gene is greater than the increase in the expression level of VEGF-A. In certain embodiments, the ratio between expression level of a gene and VEGF-A in a sample is increased as compared to the ratio of the expression levels of the gene and VEGF-A in a reference sample if there is an increase in the expression level of the gene and there is no increase in the expression level of VEGF-A. In certain embodiments, the ratio between expression level of a gene and VEGF-A in a sample is increased as compared to the ratio of the expression levels of the gene and VEGF-A in a reference sample if there is no decrease in the expression level of the gene and there is decrease in the expression level of VEGF-A. In one embodiment, the ratio between expression level of VEGF-C and VEGF-A (e.g., VEGF-C/VEGF-A) in a sample is increased as compared to the ratio of the expression levels of VEGF-C and VEGF-A in a reference sample.

In certain embodiments, the change in the ratio between the expression level of a gene and the expression level of VEGF-A in the sample as compared to the same ratio in the reference sample is a decrease. In certain embodiments, the ratio between expression level of a gene and VEGF-A in a sample is decreased as compared to the ratio of the expression levels of the gene and VEGF-A in a reference sample if the decrease in the expression level of the gene is greater than the decrease in the expression level of VEGF-A. In certain embodiments, the ratio between expression level of a gene and VEGF-A in a sample is decreased as compared to the ratio of the expression levels of the gene and VEGF-A in a reference sample if there is no increase in the expression level of the gene and there is increase in the expression level of VEGF-A. In certain embodiments, the ratio between expression level of a gene and VEGF-A in a sample is decreased as compared to the ratio of the expression levels of the gene and VEGF-A in a reference sample if there is decrease in the expression level of the gene and there is no decrease in the expression level of VEGF-A.

In certain embodiments, the samples are normalized for both differences in the amount of RNA or protein assayed and variability in the quality of the RNA or protein samples used, and variability between assay runs. Such normalization may be accomplished by measuring and incorporating the expression of certain normalizing genes, including well known housekeeping genes, such as ACTB. One or more housekeeping genes can be used to normalize the samples. Alternatively, normalization can be based on the mean or median signal of all of the assayed genes or a large subset thereof (global normalization approach). On a gene-by-gene basis, measured normalized amount of a patient tumor mRNA or protein is compared to the amount found in a reference set. Normalized expression levels for each mRNA or protein per tested tumor per patient can be expressed as a percentage of the expression level measured in the reference set. The expression level measured in a particular patient sample to be analyzed will fall at some percentile within this range, which can be determined by methods well known in the art.

In certain embodiments, relative expression level of a gene is determined as follows using one housekeeping gene:

Relative expression gene1ₛₐₘₚₗₑ₁=2 exp(Ct_{housekeeping gene}−Ct_{gene1}) with Ct determined in sample1

Relative expression gene1_{reference RNA}=2 exp(Ct_{housekeeping gene}−Ct_{gene1}) with Ct determined in the reference RNA.

Normalized relative expression gene1ₛₐₘₚₗₑ₁=(relative expression gene1ₛₐₘₚₗₑ₁/relative expression gene1_{reference RNA})×100

In certain embodiments, relative expression level of a gene is determined as follows using two housekeeping genes:

Relative expression gene1ₛₐₘₚₗₑ₁=2 exp [(Ct_{housekeeping gene1}+Ct_{housekeeping gene2})/2−Ct_{gene1}] with Ct determined in sample1

Relative expression gene1_{reference RNA}=2 exp [(Ct_{housekeeping gene1}+Ct_{housekeeping gene2})/2−Ct_{gene1}] with Ct determined in the reference RNA.

Normalized relative expression gene1ₛₐₘₚₗₑ₁=(relative expression gene1ₛₐₘₚₗₑ₁/relative expression gene1_{reference RNA})×100

Ct is the threshold cycle. The Ct is the cycle number at which the fluorescence generated within a reaction crosses the threshold line.

All experiments are normalized to a reference RNA, which is a comprehensive mix of RNA from various tissue sources (e.g., reference RNA #636538 from Clontech, Mountain View, Calif.). Identical reference RNA is included in each qRT-PCR run, allowing comparison of results between different experimental runs.

Ratio of expression between two genes is determined as:

Normalized expression gene1ₛₐₘₚₗₑ₁/Normalized expression gene2ₛₐₘₚₗₑ₁

In certain embodiments, gene1 is an angiogenic factor. In another embodiment, the angiogenic factor is VEGF-C, VEGF-D, VEGFR3 or bFGF. In one embodiment, gene2 is VEGF-A. In certain embodiments, mRNA expression of VEGF-C, VEGF-D, VEGFR3 and/or bFGF is in tumor cells.

The invention also provides methods for treating cancer in a patient comprising determining that the ratio between expression level of a gene and expression level of VEGF-A in a sample obtained from the patient has changed as compared to the ratio between the expression level of said gene and the expression level of VEGF-A in a reference sample, and administering an effective amount of an anti-cancer therapy other than a VEGF antagonist to said patient, whereby the cancer is treated. In certain embodiments, the expression level is mRNA expression level. In certain embodiments, the change in the expression level is an increase.

The invention further provides methods for treating, inhibiting or preventing relapse tumor growth or relapse cancer cell growth. Relapse tumor growth or relapse cancer cell growth is used to describe a condition in which patients undergoing or treated with one or more currently available therapies (e.g., cancer therapies, such as chemotherapy, radiation therapy, surgery, hormonal therapy and/or biological therapy/immunotherapy, anti-angiogenic therapy, anti-VEGF antibody therapy, particularly a standard therapeutic regimen for the particular cancer) is not clinically adequate to treat the patients or the patients are no longer receiving any beneficial effect from the therapy such that these patients need additional effective therapy. As used herein, the phrase can also refer to a condition of the “non-responsive/refractory” patient, e.g., which describe patients who respond to therapy yet suffer from side effects, develop resistance, do not respond to the therapy, do not respond satisfactorily to the therapy, etc. In certain embodiments, a cancer is relapse tumor growth or relapse cancer cell growth where the number of cancer cells has not been significantly reduced, or has increased, or tumor size has not been significantly reduced, or has increased, or fails any further reduction in size or in number of cancer cells. The determination of whether the cancer cells are relapse tumor growth or relapse cancer cell growth can be made either in vivo or in vitro by any method known in the art for assaying the effectiveness of treatment on cancer cells, using the art-accepted meanings of “relapse” or “refractory” or “non-responsive” in such a context. In certain embodiments, a “resistant tumor” as used herein refers to a tumor that is resistant to an anti-VEGF antibody treatment.

The invention also provides methods for treating a cell proliferative disorder in a patient comprising determining that the ratio between expression level of a gene and expression level of VEGF-A in a sample obtained from the patient has changed as compared to the ratio between the expression level of said gene and the expression level of VEGF-A in a reference sample, and administering an effective amount of an anti-cancer therapy other than a VEGF antagonist to said patient, whereby the cell proliferative disorder is treated. In certain embodiments, the expression level is mRNA expression level. In certain embodiments, the change in the expression level is an increase.

The invention also provides methods for inhibiting angiogenesis in a patient comprising determining that the ratio between expression level of a gene and expression level of VEGF-A in a sample obtained from the patient has changed as compared to the ratio between the expression level of said gene and the expression level of VEGF-A in a reference sample, and administering an effective amount of an anti-cancer therapy other than a VEGF antagonist to said patient. In certain embodiments, the expression level is mRNA expression level. In certain embodiments, the change in the expression level is an increase.

The invention also provides methods for inhibiting lymphangiogenesis in a patient comprising determining that the ratio between expression level of a gene and expression level of VEGF-A in a sample obtained from the patient has changed as compared to the ratio between the expression level of said gene and the expression level of VEGF-A in a reference sample, and administering an effective amount of an anti-cancer therapy other than a VEGF antagonist to said patient. In certain embodiments, the expression level is mRNA expression level. In certain embodiments, the change in the expression level is an increase.

In certain embodiments, the anti-cancer agent or anti-angiogenic agent is administered in combination with VEGF antagonist to treat cancer, to treat, inhibit or prevent relapse tumor growth or relapse cancer cell growth, including resistant tumor, to treat cell proliferative disorder, to inhibit angiogenesis or to inhibit lymphangiogenesis. In one embodiment, the VEGF antagonist is an anti-VEGF neutralizing antibody or fragment (e.g., humanized A4.6.1, AVASTIN® (Genentech, South San Francisco, Calif.), Y0317, M4, G6, B20, 2C3, etc.). See, e.g., U.S. Pat. Nos. 6,582,959, 6,884,879, 6,703,020; WO98/45332; WO 96/30046; WO94/10202; EP 0666868B1; U.S. Patent Applications 20030206899, 20030190317, 20030203409, and 20050112126; Popkov et al., Journal of Immunological Methods 288:149-164 (2004); and, WO2005012359. The anti-cancer agent and anti-angiogenic agent includes those known in the art and those found under the Definitions herein. In one embodiment, the anti-cancer agent or anti-angiogenic agent is anti-VEGF-C antibody. In certain embodiments, the VEGF antagonist is bevacizumab. In certain embodiments, the VEGF antagonist is administered simultaneously with the anti-cancer agent or anti-angiogenic agent. In certain embodiment, bevacizumab is administered concurrently with anti-VEGF-C antibody. In certain embodiments, bevacizumab is administered concurrently with anti-VEGF-C antibody to patients relapsed from or refractory to anti-cancer therapy comprising VEGF antagonist. In certain embodiments, the anti-cancer agent or anti-angiogenic agent is administered according to the instructions provided on the label or package insert.

A sample comprising a target gene or biomarker can be obtained by methods well known in the art, and that are appropriate for the particular type and location of the cancer of interest. See under Definitions. For instance, samples of cancerous lesions may be obtained by resection, bronchoscopy, fine needle aspiration, bronchial brushings, or from sputum, pleural fluid or blood. Genes or gene products can be detected from cancer or tumor tissue or from other body samples such as urine, sputum, serum or plasma. The same techniques discussed above for detection of target genes or gene products in cancerous samples can be applied to other body samples. Cancer cells may be sloughed off from cancer lesions and appear in such body samples. By screening such body samples, a simple early diagnosis can be achieved for these cancers. In addition, the progress of therapy can be monitored more easily by testing such body samples for target genes or gene products.

Means for enriching a tissue preparation for cancer cells are known in the art. For example, the tissue may be isolated from paraffin or cryostat sections. Cancer cells may also be separated from normal cells by flow cytometry or laser capture microdissection. These, as well as other techniques for separating cancerous from normal cells, are well known in the art. If the cancer tissue is highly contaminated with normal cells, detection of signature gene or protein expression profile may be more difficult, although techniques for minimizing contamination and/or false positive/negative results are known, some of which are described herein below. For example, a sample may also be assessed for the presence of a biomarker known to be associated with a cancer cell of interest but not a corresponding normal cell, or vice versa.

In certain embodiments, the expression of proteins in a sample is examined using immunohistochemistry (“IHC”) and staining protocols. Immunohistochemical staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods.

The tissue sample may be fixed (i.e. preserved) by conventional methodology (See e.g., “Manual of Histological Staining Method of the Armed Forces Institute of Pathology,” 3^{rd} edition (1960) Lee G. Luna, HT (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). One of skill in the art will appreciate that the choice of a fixative is determined by the purpose for which the sample is to be histologically stained or otherwise analyzed. One of skill in the art will also appreciate that the length of fixation depends upon the size of the tissue sample and the fixative used. By way of example, neutral buffered formalin, Bouin's or paraformaldehyde, may be used to fix a sample.

Generally, the sample is first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology (See e.g., “Manual of Histological Staining Method of the Armed Forces Institute of Pathology”, supra). Examples of paraffin that may be used include, but are not limited to, Paraplast, Broloid, and Tissuemay. Once the tissue sample is embedded, the sample may be sectioned by a microtome or the like (See e.g., “Manual of Histological Staining Method of the Armed Forces Institute of Pathology”, supra). By way of example for this procedure, sections may range from about three microns to about five microns in thickness. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used (See e.g., “Manual of Histological Staining Method of the Armed Forces Institute of Pathology”, supra). Alternatively, commercially available deparaffinizing non-organic agents such as Hemo-De7 (CMS, Houston, Tex.) may be used.

In certain embodiments, subsequent to the sample preparation, a tissue section may be analyzed using IHC. IHC may be performed in combination with additional techniques such as morphological staining and/or fluorescence in-situ hybridization. Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.

(b) Colloidal gold particles.

(c) Fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above. The fluorescent labels can be conjugated to the antibody using the techniques disclosed in Current Protocols in Immunology, supra, for example. Fluorescence can be quantified using a fluorimeter.

(d) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed. J. Langone & H. Van Vunakis), Academic press, New York, 73:147-166 (1981).

Examples of enzyme-substrate combinations include, for example:

(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));

(ii) alkaline phosphatase (AP) with para-Nitrophenyl phosphate as chromogenic substrate; and

(iii) β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase).

Numerous other enzyme-substrate combinations are available to those skilled in the art. For a general review of these, see U.S. Pat. Nos. 4,275,149 and 4,318,980. Sometimes, the label is indirectly conjugated with the antibody. The skilled artisan will be aware of various techniques for achieving this. For example, the antibody can be conjugated with biotin and any of the four broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Aside from the sample preparation procedures discussed above, further treatment of the tissue section prior to, during or following IHC may be desired. For example, epitope retrieval methods, such as heating the tissue sample in citrate buffer may be carried out (see, e.g., Leong et al. Appl. Immunohistochem. 4(3):201 (1996)).

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation. The extent of binding of antibody to the sample is determined by using any one of the detectable labels discussed above. In certain embodiments, the label is an enzymatic label (e.g. HRPO) which catalyzes a chemical alteration of the chromogenic substrate such as 3,3′-diaminobenzidine chromogen. In one embodiment, the enzymatic label is conjugated to antibody which binds specifically to the primary antibody (e.g. the primary antibody is rabbit polyclonal antibody and secondary antibody is goat anti-rabbit antibody).

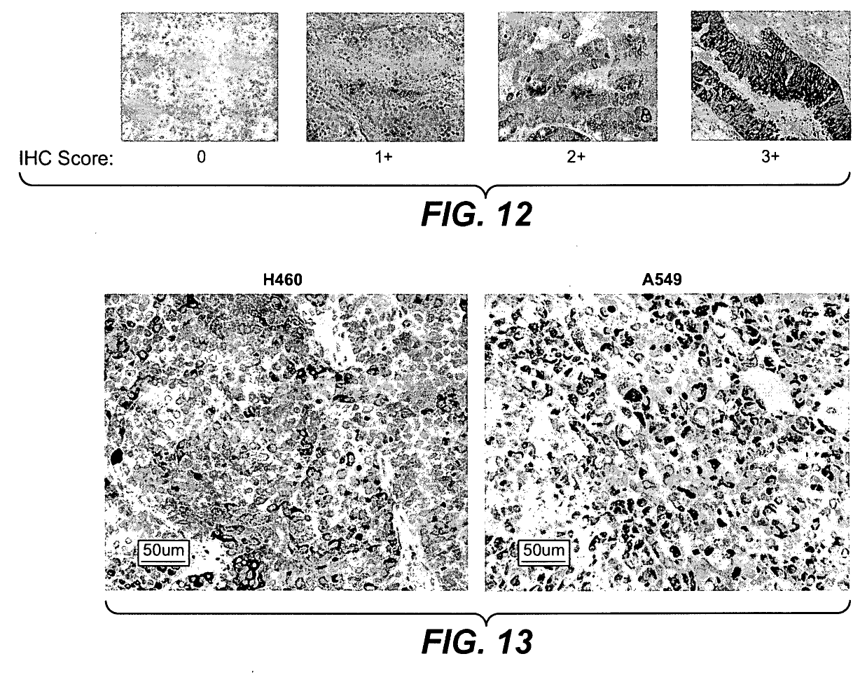

Specimens thus prepared may be mounted and coverslipped. Slide evaluation is then determined, e.g., using a microscope, and staining intensity criteria, routinely used in the art, may be employed. Staining intensity criteria may be evaluated as follows (see FIG. 12):

TABLE 2

| Staining Pattern | Score |
|---|---|
| No staining is observed in cells. | 0 |
| Faint/barely perceptible staining is detected in more | 1+ |
| than 10% of the cells. | |
| Weak to moderate staining is observed in more than | 2+ |
| 10% of the cells. | |
| Moderate to strong staining is observed in more than | 3+ |
| 10% of the cells. | |

In some embodiments, a staining pattern score of about 1+ or higher is diagnostic and/or prognostic. In certain embodiments, a staining pattern score of about 2+ or higher in an IHC assay is diagnostic and/or prognostic. In other embodiments, a staining pattern score of about 3 or higher is diagnostic and/or prognostic. In one embodiment, it is understood that when cells and/or tissue from a tumor or colon adenoma are examined using IHC, staining is generally determined or assessed in tumor cell and/or tissue (as opposed to stromal or surrounding tissue that may be present in the sample).

In alternative methods, the sample may be contacted with an antibody specific for said biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, -galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

It is contemplated that the above described techniques may also be employed to detect expression of one or more of the target genes wherein the target genes are antiangiogenic factors as defined herein. In certain embodiments, the target genes are VEGF-A, VEGF-C, VEGF-D, bFGF and/or VEGFR3.

Methods of the invention further include protocols which examine the presence and/or expression of mRNAs of the one or more target genes, including, but not limited to, VEGF-A, VEGF-C, VEGF-D, bFGF and VEGFR3, in a tissue or cell sample. Methods for the evaluation of mRNAs in cells are well known and include, for example, hybridization assays using complementary DNA probes (such as in situ hybridization using labeled riboprobes specific for the one or more genes, including, but not limited to, VEGF-A, VEGF-C, VEGF-D, bFGF and VEGFR3, Northern blot and related techniques) and various nucleic acid amplification assays (such as RT-PCR using complementary primers specific for one or more of the genes, and other amplification type detection methods, such as, for example, branched DNA, SISBA, TMA and the like).

Tissue or cell samples from mammals can be conveniently assayed for mRNAs using Northern, dot blot or PCR analysis. For example, RT-PCR assays such as quantitative PCR assays are well known in the art. In an illustrative embodiment of the invention, a method for detecting a target mRNA in a biological sample comprises producing cDNA from the sample by reverse transcription using at least one primer; amplifying the cDNA so produced using a target polynucleotide as sense and antisense primers to amplify target cDNAs therein; and detecting the presence of the amplified target cDNA. In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined.

Optional methods of the invention include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlate with increased or reduced clinical benefit of anti-angiogenic therapy may be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment. (see, e.g., WO 01/75166 published Oct. 11, 2001; (see, for example, U.S. Pat. No. 5,700,637, U.S. Pat. No. 5,445,934, and U.S. Pat. No. 5,807,522, Lockart, Nature Biotechnology, 14:1675-1680 (1996); Cheung, V. G. et al., Nature Genetics 21(Suppl):15-19 (1999) for a discussion of array fabrication). DNA microarrays are miniature arrays containing gene fragments that are either synthesized directly onto or spotted onto glass or other substrates. Thousands of genes are usually represented in a single array. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. Currently two main types of DNA microarrays are being used: oligonucleotide (usually 25 to 70 mers) arrays and gene expression arrays containing PCR products prepared from cDNAs. In forming an array, oligonucleotides can be either prefabricated and spotted to the surface or directly synthesized on to the surface (in situ).

The Affymetrix GeneChip® system is a commercially available microarray system which comprises arrays fabricated by direct synthesis of oligonucleotides on a glass surface. Probe/Gene Arrays: Oligonucleotides, usually 25 mers, are directly synthesized onto a glass wafer by a combination of semiconductor-based photolithography and solid phase chemical synthesis technologies. Each array contains up to 400,000 different oligos and each oligo is present in millions of copies. Since oligonucleotide probes are synthesized in known locations on the array, the hybridization patterns and signal intensities can be interpreted in terms of gene identity and relative expression levels by the Affymetrix Microarray Suite software. Each gene is represented on the array by a series of different oligonucleotide probes. Each probe pair consists of a perfect match oligonucleotide and a mismatch oligonucleotide. The perfect match probe has a sequence exactly complimentary to the particular gene and thus measures the expression of the gene. The mismatch probe differs from the perfect match probe by a single base substitution at the center base position, disturbing the binding of the target gene transcript. This helps to determine the background and nonspecific hybridization that contributes to the signal measured for the perfect match oligo. The Microarray Suite software subtracts the hybridization intensities of the mismatch probes from those of the perfect match probes to determine the absolute or specific intensity value for each probe set. Probes are chosen based on current information from Genbank and other nucleotide repositories. The sequences are believed to recognize unique regions of the 3′ end of the gene. A GeneChip Hybridization Oven (“rotisserie” oven) is used to carry out the hybridization of up to 64 arrays at one time. The fluidics station performs washing and staining of the probe arrays. It is completely automated and contains four modules, with each module holding one probe array. Each module is controlled independently through Microarray Suite software using preprogrammed fluidics protocols. The scanner is a confocal laser fluorescence scanner which measures fluorescence intensity emitted by the labeled cRNA bound to the probe arrays. The computer workstation with Microarray Suite software controls the fluidics station and the scanner. Microarray Suite software can control up to eight fluidics stations using preprogrammed hybridization, wash, and stain protocols for the probe array. The software also acquires and converts hybridization intensity data into a presence/absence call for each gene using appropriate algorithms. Finally, the software detects changes in gene expression between experiments by comparison analysis and formats the output into .txt files, which can be used with other software programs for further data analysis.

Expression of a selected gene or biomarker in a tissue or cell sample may also be examined by way of functional or activity-based assays. For instance, if the biomarker is an enzyme, one may conduct assays known in the art to determine or detect the presence of the given enzymatic activity in the tissue or cell sample.

The kits of the invention have a number of embodiments. In certain embodiments, a kit comprises a container, a label on said container, and a composition contained within said container; wherein the composition includes one or more primary antibodies that bind to one or more target polypeptide sequences corresponding to one or more of target genes including, but not limited to, VEGF-A, VEGF-C, VEGF-D, bFGF and VEGFR3, the label on the container indicating that the composition can be used to evaluate the presence of one or more target proteins in at least one type of mammalian cell, and instructions for using the antibodies for evaluating the presence of one or more target proteins in at least one type of mammalian cell. The kit can further comprise instructions for measuring expression levels of one or more target proteins to calculate a ratio between expression levels of two target proteins. In one embodiment, one of the target protein is VEGF-A. In another embodiment, the second target protein is VEGF-C, VEGF-D, bFGF or VEGFR3. The kit can further comprise a set of instructions and materials for preparing a tissue sample and applying antibody and probe to the same section of a tissue sample. The kit may include both a primary and secondary antibody, wherein the secondary antibody is conjugated to a label, e.g., an enzymatic label.

Another embodiment is a kit comprising a container, a label on said container, and a composition contained within said container; wherein the composition includes one or more polynucleotides that hybridize to the polynucleotide sequence of the one or more genes including, but not limited to, VEGF-A, VEGF-C, VEGF-D, bFGF and/or VEGFR3, under stringent conditions, the label on said container indicates that the composition can be used to evaluate the presence of and/or expression levels of the one or more target genes including, but not limited to, VEGF-A, VEGF-C, VEGF-D. bFGF and/or VEGFR3, in at least one type of mammalian cell, and instructions for using the polynucleotide for evaluating the presence of and/or expression levels of one or more target RNAs or DNAs in at least one type of mammalian cell. The kit can further comprise instructions for calculating a ratio between expression levels of two target genes.

Other optional components in the kit include one or more buffers (e.g., block buffer, wash buffer, substrate buffer, etc), other reagents such as substrate (e.g., chromogen) which is chemically altered by an enzymatic label, epitope retrieval solution, control samples (positive and/or negative controls), control slide(s) etc.

Although in the foregoing description the invention is illustrated with reference to certain embodiments, it is not so limited. Indeed, various modifications of the invention in addition to those shown and described herein will become apparent to those skilled in the art from the foregoing description and fall within the scope of the appended claims. All references cited throughout the specification, and the references cited therein, are hereby expressly incorporated by reference in their entirety.

### EXAMPLES

### Example 1

### In Vivo Studies

All studies were conducted in accordance with the Guide for the Care and Use of Laboratory Animals, published by the NIH (NIH Publication 85-23, revised 1985). An Institutional Animal Care and Use Committee (IACUC) approved all animal protocols.

Following human tumor model studies were conducted at Piedmont Research Center, LLC (Morrisville, N.C.) using standardized techniques: A549, H460, MV522, MDA-MB231, DLD-1, BxPC3, HT29, SKMES, PC3. Human tumor cells were implanted subcutaneously in the right flank of each test mouse. For example, for H460, xenografts were initiated from cultured H460 human non-small cell lung carcinoma cells (grown to mid-log phase in RPMI-1640 medium containing 10% heat-inactivated fetal bovine serum, 100 units/mL penicillin G, 100 μg/mL streptomycin sulfate, 0.25 μg/mL amphotericin B, 1 mM sodium pyruvate, 2 mM glutamine, 10 mM HEPES, 0.075% sodium bicarbonate, and 25 μg/mL gentamicin) or from A549 human lung adenocarcinoma cells (cultured in Kaighn's modified Ham's F12 medium containing 10% heat-inactivated fetal bovine serum, 100 units/mL penicillin G, 100 μg/mL streptomycin sulfate, 0.25 μg/mL amphotericin B, 2 mM glutamine, 1 mM sodium pyruvate, and 25 μg/mL gentamicin). On the day of tumor implant, H460 cells were harvested and resuspended in PBS at a concentration of 5×10⁷ cells/mL. Each test mouse received 1×10⁷H460 tumor cells implanted subcutaneously in the right flank. For A549 tumors, A549 cells were resuspended in 100% Matrigel™ matrix (BD Biosciences, San Jose, Calif.) at a concentration of 5×10⁷ cells/mL. A549 cells (1×10⁷ in 0.2 mL) were implanted subcutaneously in the right flank of each test mouse, and tumor growth was monitored.

Tumor growth was monitored as the average size approached 120-180 mm³. On study day 1, individual tumors sizes ranged from 126 to 196 mm³ and the animals were sorted by tumor size into three treatment and control groups. Tumor volume was calculated using the formula:

Tumor volume (mm³)==(w²×l)/2

where w=width and l=length in mm of the tumor.

All treatments were administered intra-peritoneally. Tumors were treated twice weekly for up to 10-20 weeks with 5-10 mg/kg each of control antibody, an agent blocking VEGF-A activity (anti-VEGF-A antibody B20-4.1 at 5 mg/kg), or the combination of the two agents blocking VEGF-A and VEGF-C activity (anti-VEGF-C antibody at 10 mg/kg). For the combination treatment group, anti-VEGF-C antibody was administered no later than thirty minutes after anti-VEGF-A antibody. Each dose was delivered in a volume of 0.2 mL per 20 grams body weight (10 mL/kg), and was scaled to the body weight of the animal.

Tumor volume was recorded twice weekly using calipers. Each animal was euthanized when its tumor reached the endpoint size (generally 1000 mm³) or at the conclusion of the study, whichever came first.

The time to endpoint (TTE) was calculated from the following equation:

TTE(days)=(log₁₀(endpoint volume, mm³−b)/m

where b is the intercept and m is the slope of the line obtained by linear regression of a log-transformed tumor growth data set.

Animals that do not reach the endpoint are assigned a TTE value equal to the last day of the study. Animals classified as NTR (non-treatment-related) deaths due to accident (NTRa) or due unknown causes (NTRu) are excluded from TTE calculations (and all further analyses). Animals classified as TR (treatment-related) deaths or NTRm (non-treatment-related death due to metastasis) are assigned a TTE value equal to the day of death.

Treatment outcome was evaluated by tumor growth delay (TGD), which is defined as the increase in the median time to endpoint (TTE) in a treatment group compared to the control group, which was calculated as follows:

TGD=T−C,

expressed in days, or as a percentage of the median TTE of the control group, which was calculated as follows:

% TGD=[(T−C)/C]×100,

where T=median TTE for a treatment group and C=median TTE for the control group.

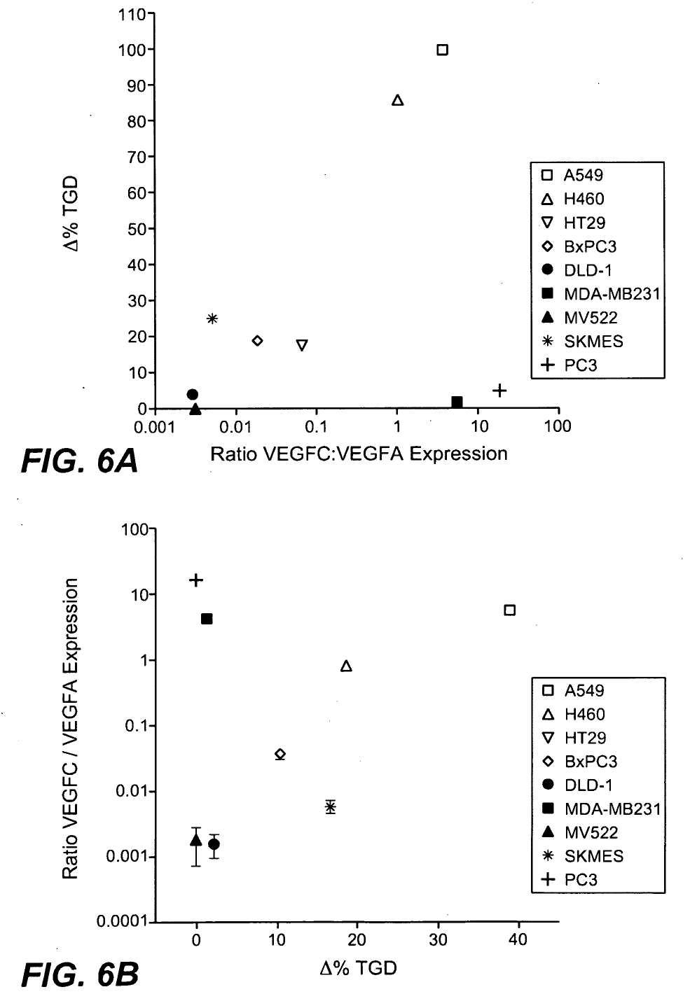

The Δ % TGD was calculated following the equation Δ % TGD=% TGD2−% TGD1=([(T2−C)/C]×100, where C=median TTE in the group receiving a control agent, and T2=median TTE in the group receiving the two agents blocking VEGF-A and VEGF-C) minus ([(T1−C)/C]×100, where C=median TTE in the group receiving a control agent, and T1=median TTE in the group receiving the agents blocking VEGF-A alone). See e.g., FIGS. 6A, 7A and 14A.

Alternatively, the Δ % TGD was calculated following the equation % TGD=[(T−C)/C]×100, where C=median TTE in the group receiving the agent blocking VEGF-A alone, and T=median TTE in the group receiving the two agents blocking VEGF-A and VEGF-C. See e.g., FIGS. 6B, 7B, 8 and 14B.

In some studies efficacy was calculated as percent tumor growth inhibition (% TGI), which was calculated as follows:

% TGI=[(median tumor volume control−median tumor volume treated)/median tumor volume control]×100

The Δ % TGI was calculated as the difference in % TGI between the group receiving the two agents blocking VEGF-A and VEGF-C, and the group receiving the agent blocking VEGF-A alone.

Tumor were harvested and fixated overnight in 10% NBF, followed by 70% EtOH and subsequent embedding in paraffin.

Anti-VEGF-C antibody treatment resulted in delay in tumor progression in H460 and A549 tumors when combined with anti-VEGF-A treatment (FIGS. 6, 7 and 8).

### Example 2

### IHC

Microtome sections from paraffin embedded tumors were stained for VEGF-C using a commercial antibody (IBL, Japan). In brief, sections were deparaffinized, hydrated and subjected to Target Retrieval (Dako, Glostrup, Denkmark). Endogenous peroxidase activity (KPL Inc., Gaithersburg, Md.) and avidin/biotin reactivity (Vector Laboratories, Burlingame, Calif.) were blocked according to manufacturers protocol. Endogenous immunoglobulins were blocked by incubation with 10% goat serum in 3% BSA/PBS (‘blocking serum’) for 30 min at room temperature (RT). Anti-VEGF-C antibody was diluted to 0.5 μg/ml in blocking serum and incubated with the section for 1 h at RT. Sections incubated with rabbit IgG were used as negative control. After washes in TRIS buffered saline, sections were incubated with biotinylated goat anti-rabbit antibody (Vector Laboratories, Burlingame, Calif.) at 7.5 μg/ml in blocking serum for 30 min at RT. Sections were washed, followed by incubation with peroxidase coupled avidin reagent (Vector Laboratories, Burlingame, Calif.) for 30 min at RT. Following additional washes, bound antibody was detected using the peroxidase substrate DAB (Pierce, Rockford, Ill.). Sections were washed, coversliped and subjected to IHC scoring. IHC score was determined as follows:

| Staining Pattern | Score |
|---|---|
| No staining is observed in cells. | 0 |
| Faint/barely perceptible staining is detected in more | 1+ |
| than 10% of the cells. | |
| Weak to moderate staining is observed in more than | 2+ |
| 10% of the cells. | |
| Moderate to strong staining is observed in more than | 3+ |
| 10% of the cells. | |

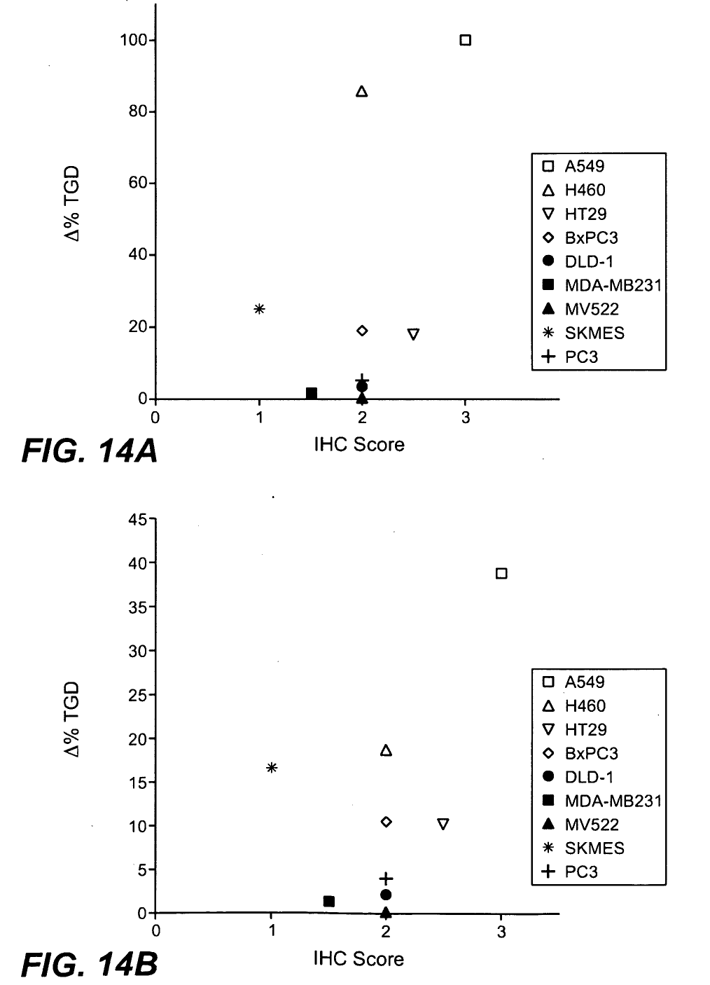

Representative images of stained sections of xenograft tumors showed that H460 and A549 are positive for VEGF-C expression by IHC (FIG. 13). These results indicate that there is a correlation between IHC score and delay in tumor progression (Δ % TGD) for H460 and A549 tumor models (FIG. 14).

### Example 3

### RNA isolation

Two 5 μm thick microtome sections were cut from human xenograft formalin-fixed, paraffin embedded tumors and subjected to RNA isolation (Roche Applied Sciences, Indianapolis, Ind.). In brief, paraffin wax was solubilized in 900 μl Envirene (Hardy Diagnostics, Santa Maria, Calif.), and remaining tissue fragments precipitated after the addition of 450 μl ethanol. Pellets were air dried, and digested overnight at 55° C. with Proteinase K working solution according to manufacturer's protocol. Following column purification, the sample was digested with DNase for 45 minutes at 37° C. to remove genomic DNA which would interfere with the subsequent analysis. DNase was removed by Proteinase K digestion at 55° C. for 1 hour, followed by column purification. The sample was eluted in a total of 50 μl elution buffer, centrifuged to precipitate column residues, and transferred to a new reaction tube. RNA concentrations were assessed using a spectrophotometer or a bioanalyzer (Agilent, Foster City, Calif.), and 50 ng of total RNA used per reaction in the subsequent gene expression analysis.

qRT-PCR

Gene specific primer and probe sets were designed for qRT-PCR expression analysis of 18SrRNA and RPS13 (housekeeping genes), and human VEGF-A, human VEGF-C, human VEGF-D, human bFGF and human VEGFR3.

| Gene | | Assay |
|---|---|---|
| assay | Sequence | sensitivity * |
| 18SrRNA | AGT CCC TGC CCT TTG TAC ACA | 7 |
| | (SEQ ID NO: 1) | |
| | CCG AGG GCC TCA CTA AAC C | |
| | (SEQ ID NO: 2) | |
| | CGC CCG TCG CTA CTA CCG ATT | |
| | GG | |
| | (SEQ ID NO:3) | |
| RPS13 | CACCGTTTGGCTCGATATTA | 18.8 |
| | (SEQ ID NO: 4) | |
| | GGCAGAGGCTGTAGATGATTC | |
| | (SEQ ID NO: 5) | |
| | ACCAAGCGAGTCCTCCCTCCC | |
| | (SEQ ID NO: 6) | |
| bFGF | ACCCCGACGGCCGA | 24.8 |
| | (SEQ ID NO: 7) | |
| | TCTTCTGCTTGAAGTTGTAGCTTGA | |
| | (SEQ ID NO: 8) | |
| | TCCGGGAGAAGAGCGACCCTCAC | |
| | (SEQ ID NO: 9) | |
| VEGF-A | GCA GAA TCA TCA CGA AGT GG | 23.7 |
| | (SEQ ID NO: 10) | |
| | TCT CGA TTG GAT GGC AGT AG | |
| | (SEQ ID NO: 11) | |
| | TGC GCT GAT AGA CAT CCA TGA | |
| | ACT TCA | |
| | (SEQ ID NO: 12) | |
| VEGF-C | CAGTGTCAGGCAGCGAACAA | 27.9 |
| | (SEQ ID NO: 13) | |
| | CTTCCTGAGCCAGGCATCTG | |
| | (SEQ ID NO: 14) | |
| | CTGCCCCACCAATTACATGTGGAATAAT | |
| | CA | |
| | (SEQ ID NO: 15) | |
| VEGF-D | CTGCCAGAAGCACAAGCTAT | 25.1 |
| | (SEQ ID NO: 16) | |
| | ACATGGTCTGGTATGAAAGGG | |
| | (SEQ ID NO: 17) | |
| | CACCCAGACACCTGCAGCTGTG | |
| | (SEQ ID NO: 18) | |
| VEGFR3 | ACAGACAGTGGGATGGTGCTGGCC | 25.6 |
| | (SEQ ID NO: 19) | |
| | CAAAGGCTCTGTGGACAACCA | |
| | (SEQ ID NO: 20) | |
| | TCTCTATCTGCTCAAACTCCTCCG | |
| | (SEQ ID NO: 21) | |

* average Ct on 50 ng reference RNA (#636538; Clontech, Mountain View, CA)

For example, in one embodiment, relative expression level of VEGF-C was calculated as follows:

Relative expression VEGF-Cₛₐₘₚₗₑ=2 exp(Ct_{18SrRNA}−Ct_{VEGF-C}) with Ct determined in the sample, where Ct is the threshold cycle.

In another embodiment, relative expression level of VEGF-C was calculated as follows:

Relative expression VEGF-Cₛₐₘₚₗₑ=2 exp [(Ct_{18SrRNA}+Ct_{RPS13})/2−Ct_{VEGF-C}) with Ct determined in the sample, where Ct is the threshold cycle.

The Ct is the cycle number at which the fluorescence generated within a reaction crosses the threshold line.

To allow comparison of results from different reaction plates, relative expression was then calculated as a fraction to the relative expression to an internal reference RNA that was identical in all experimental runs, multiplied by 100:

Normalized relative expression VEGF-Cₛₐₘₚₗₑ=(relative expression VEGF-Cₛₐₘₚₗₑ/relative expression VEGF-C_{reference RNA})×100, where relative expression VEGF-C_{reference RNA}=2 exp(Ct_{18SrRNA}−Ct_{VEGF-C}) with Ct determined in the reference RNA

Using this calculation, samples that had any signal in the qRT-PCR reaction had values above ‘1’, samples with values below ‘1’ are negative for the particular analyte.

The ratio between the relative expression levels of VEGF-C and VEGF-A were then calculated as follows:

Normalized expression VEGF-C/Normalized expression VEGF-A

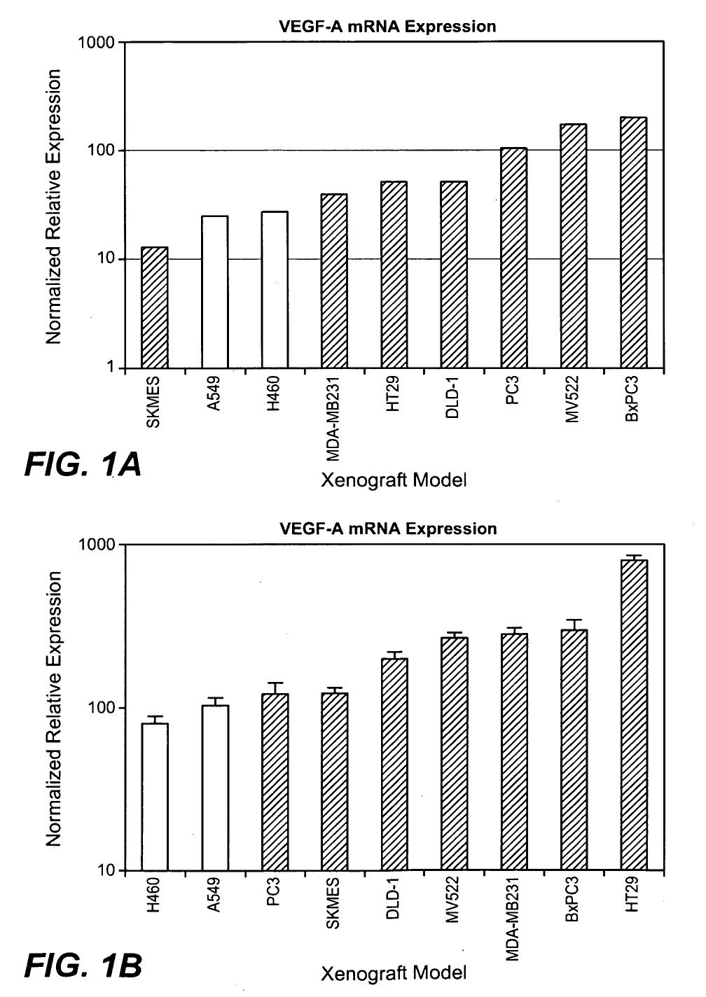

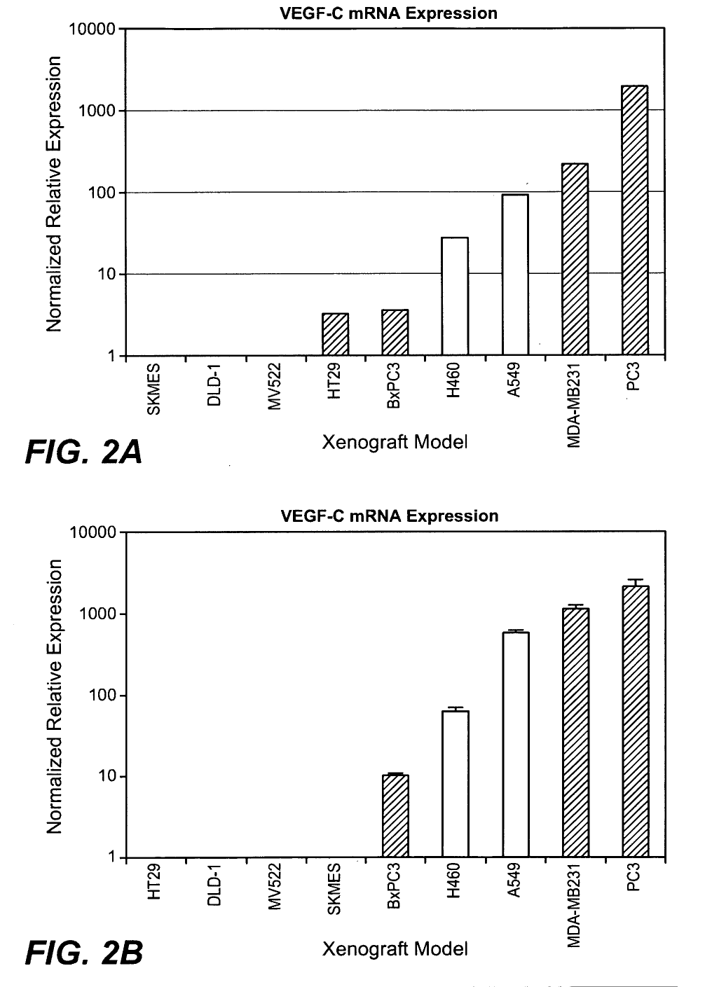

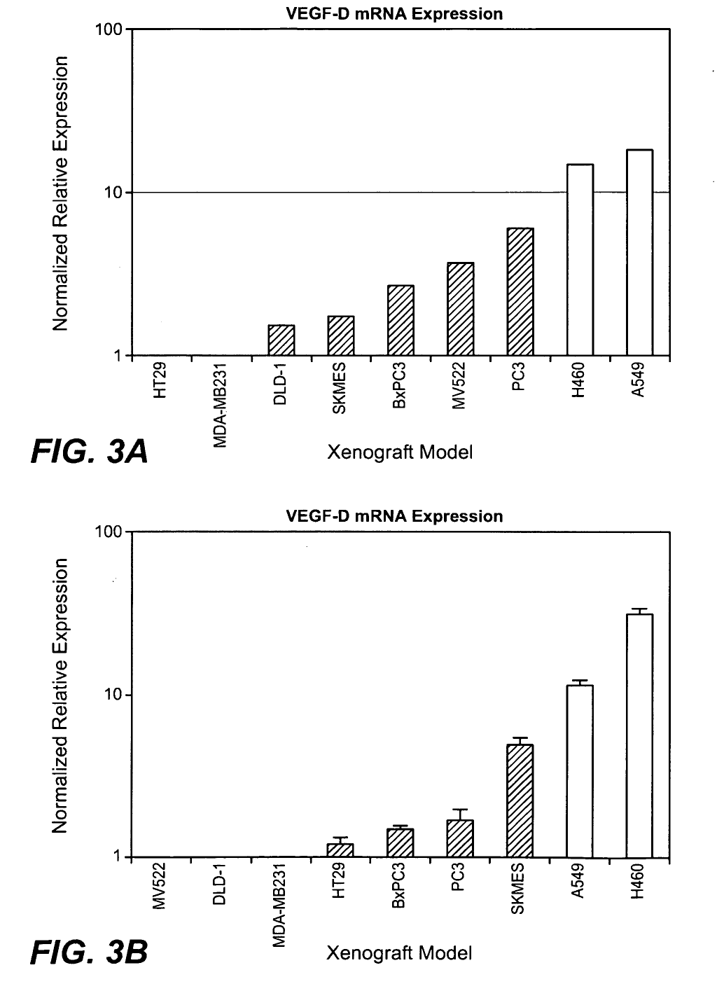

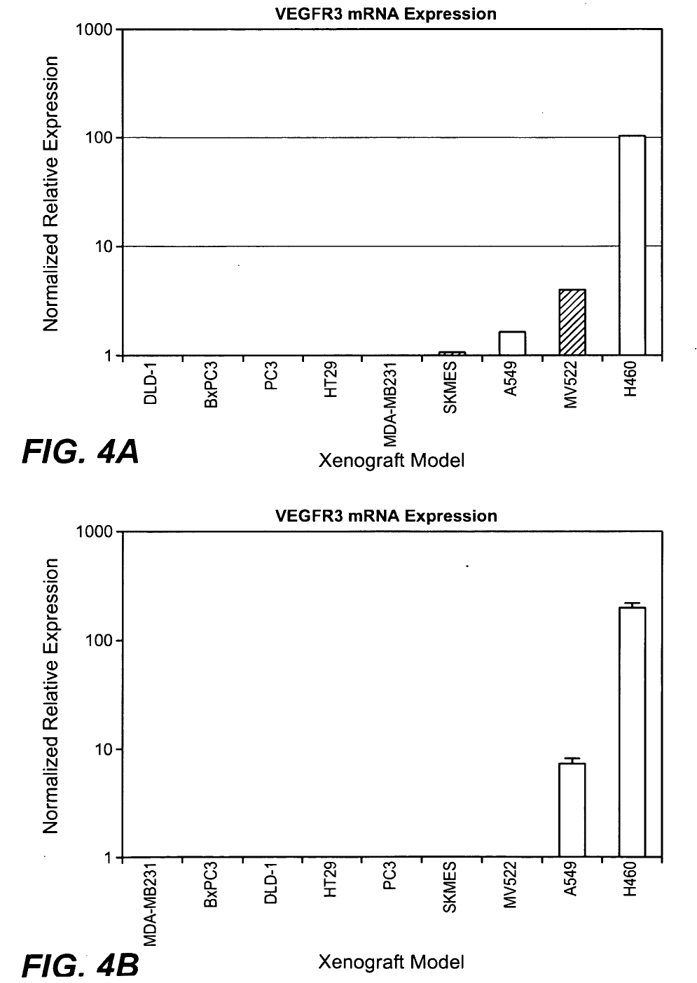

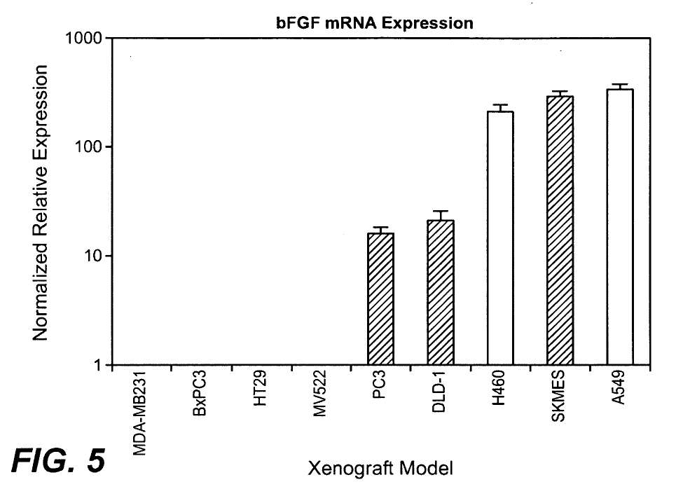

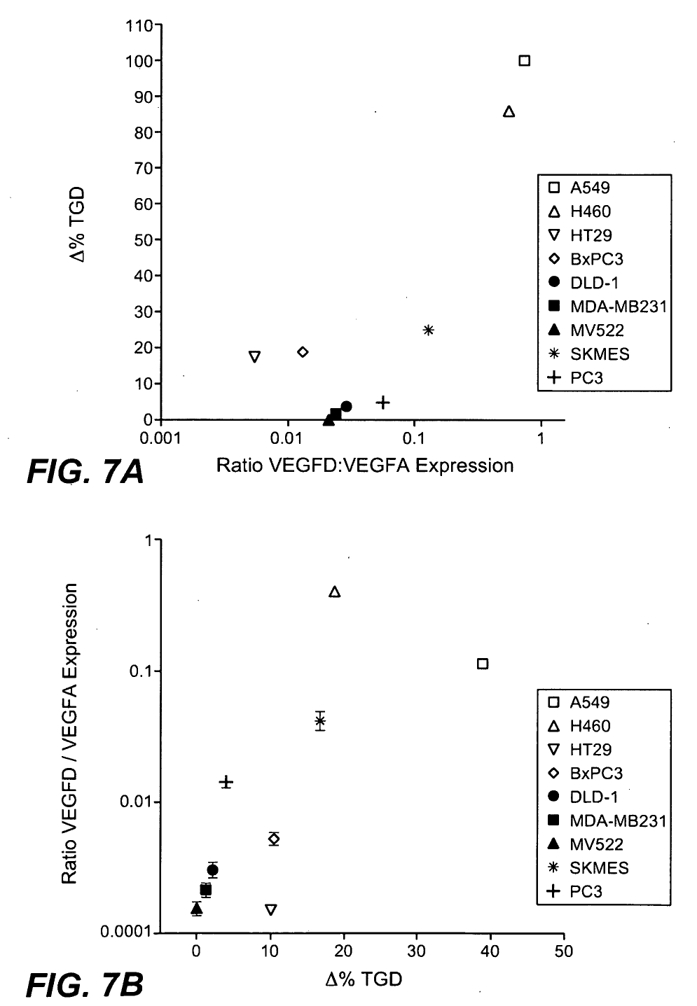

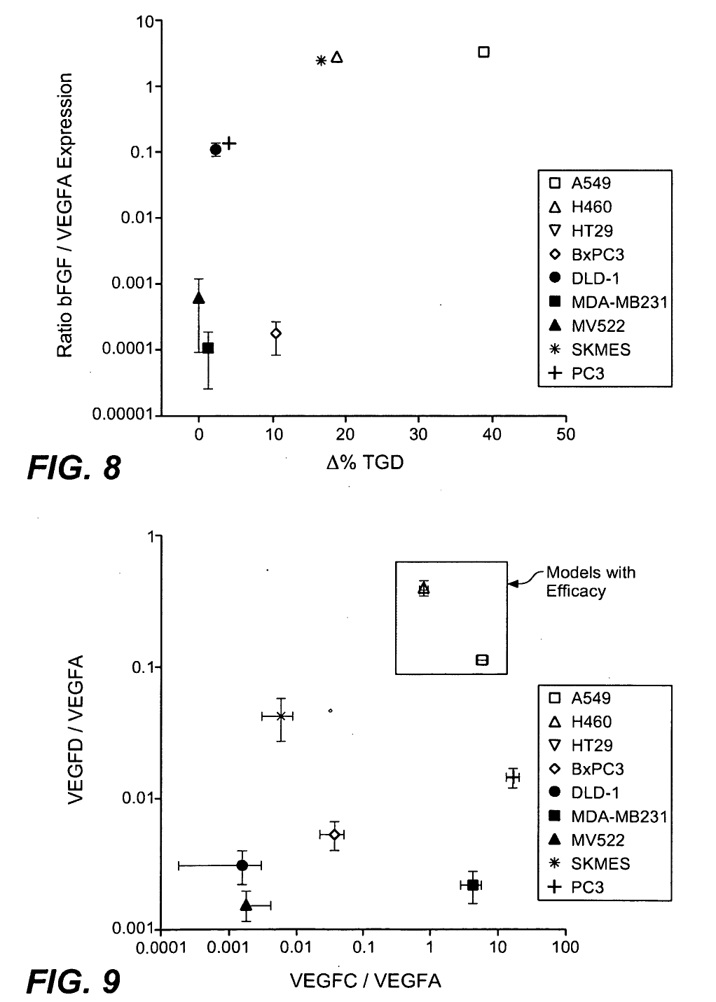

The gene expression analysis indicates that H460 and A549 were in the lower range of VEGF-A expression range in tumor models, while they were in the higher range of the VEGF-C, VEGF-D and bFGF expression range (FIGS. 1-3 and 5). In addition, these models were positive for tumor cell expression of VEGFR3 (FIG. 4). These results suggest a correlation between an increase in efficacy as a result of combined treatment with anti-VEGF-C and anti-VEGF-A antibodies and higher ratio of VEGF-C/VEGF-A normalized relative expression levels (FIG. 6), a correlation between an increase in efficacy as a result of combined treatment with anti-VEGF-C and anti-VEGF-A antibodies and higher ratio of VEGF-D/VEGF-A normalized relative expression levels (FIG. 7), a correlation between an increase in efficacy as a result of combined treatment with anti-VEGF-C and anti-VEGF-A antibodies and higher ratio of bFGF/VEGF-A normalized relative expression levels (FIG. 8), and/or a correlation between an increase in efficacy as a result of combined treatment with anti-VEGF-C and anti-VEGF-A antibodies and presence of VEGFR3 expression in the tumor cells (FIG. 4).

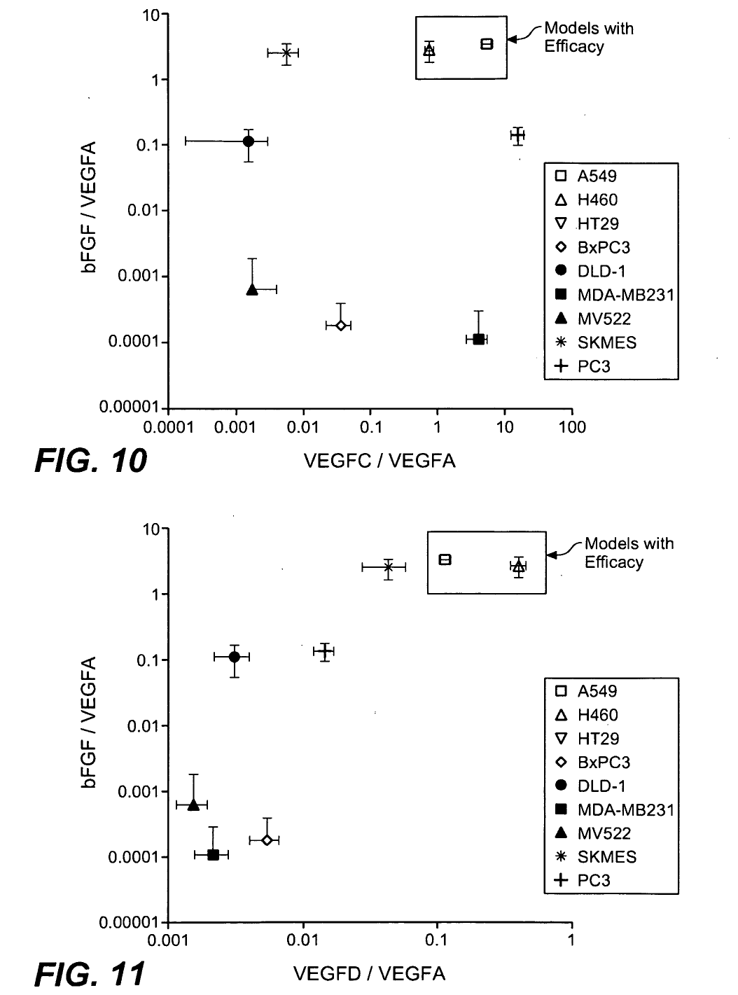

The gene expression analysis further suggest a correlation between an increase in efficacy as a result of combined treatment with anti-VEGF-C and anti-VEGF-A antibodies and higher ratio of VEGF-C/VEGF-A and VEGF-D/VEGF-A normalized relative expression levels (FIG. 9), a correlation between an increase in efficacy as a result of combined treatment with anti-VEGF-C and anti-VEGF-A antibodies and higher ratio of bFGF/VEGF-A and VEGF-C/VEGF-A normalized relative expression levels (FIG. 10), and/or a correlation between an increase in efficacy as a result of combined treatment with anti-VEGF-C and anti-VEGF-A antibodies and higher ratio of bFGF/VEGF-A and VEGF-D/VEGF-A normalized relative expression levels (FIG. 11).

What is claimed:

1. A method of identifying a patient who may benefit from anti-cancer therapy in addition to VEGF antagonist, comprising determining the ratio between expression level of a gene and expression level of VEGF-A in a sample obtained from the patient, wherein a change in the ratio between the expression level of said gene and the expression level of VEGF-A in the sample as compared to the ratio between the expression level of said gene and the expression level of VEGF-A in a reference sample indicates that the patient may benefit from anti-cancer therapy in addition to VEGF antagonist.

2. The method of claim 1, wherein the change in the ratio is an increase.

3. The method of claim 1, wherein the change in the ratio is a decrease.

4. The method of claim 1, wherein the expression level is mRNA expression level.

5. The method of claim 1, wherein the expression level is protein expression level.

6. The method of claim 1, wherein said gene is an angiogenic factor.

7. The method of claim 6, wherein the expression level is mRNA expression level.

8. The method of claim 7, wherein the mRNA expression level is measured using qRT-PCR or qPCR.

9. The method of claim 7, wherein the change in the ratio between the mRNA expression level of said angiogenic factor and the mRNA expression level of VEGF-A is an increase.

10. The method of claim 6, wherein said angiogenic factor is VEGF-C.

11. The method of claim 10 further comprising determining the ratio between mRNA expression level of VEGF-D and mRNA expression level of VEGF-A in the sample, wherein the ratio between mRNA expression level of VEGF-D and mRNA expression level of VEGF-A in the sample is increased as compared to the ratio between mRNA expression level of VEGF-D and mRNA expression level of VEGF-A in the reference sample.

12. The method of claim 10 further comprising determining the ratio between mRNA expression level of bFGF2 and mRNA expression level of VEGF-A in the sample, wherein the ratio between mRNA expression level of bFGF2 and mRNA expression level of VEGF-A in the sample is increased as compared to the ratio between mRNA expression level of bFGF2 and mRNA expression level of VEGF-A in the reference sample.

13. The method of claim 6, wherein said angiogenic factor is VEGF-D.

14. The method of claim 13 further comprising determining the ratio between mRNA expression level of VEGF-C and mRNA expression level of VEGF-A in the sample, wherein the ratio between mRNA expression level of VEGF-C and mRNA expression level of VEGF-A in the sample is increased as compared to the ratio between mRNA expression level of VEGF-C and mRNA expression level of VEGF-A in the reference sample.

15. The method of claim 13 further comprising determining the ratio between mRNA expression level of bFGF2 and mRNA expression level of VEGF-A in the sample, wherein the ratio between mRNA expression level of bFGF2 and mRNA expression level of VEGF-A in the sample is increased as compared to the ratio between mRNA expression level of bFGF2 and mRNA expression level of VEGF-A in the reference sample.

16. The method of claim 6, wherein said angiogenic factor is bFGF.

17. The method of claim 16 further comprising determining the ratio between mRNA expression level of VEGF-C and mRNA expression level of VEGF-A in the sample, wherein the ratio between mRNA expression level of VEGF-C and mRNA expression level of VEGF-A in the sample is increased as compared to the ratio between mRNA expression level of VEGF-C and mRNA expression level of VEGF-A in the reference sample.

18. The method of claim 16 further comprising determining the ratio between mRNA expression level of VEGF-D and mRNA expression level of VEGF-A in the sample, wherein the ratio between mRNA expression level of VEGF-D and mRNA expression level of VEGF-A in the sample is increased as compared to the ratio between mRNA expression level of VEGF-D and mRNA expression level of VEGF-A in the reference sample.

19. The method of claim 1, wherein the VEGF antagonist is anti-VEGF antibody.

20. The method of claim 19, wherein the anti-VEGF antibody is bevacizumab.

21. The method of claim 19 further comprising administering to the patient an effective amount of anti-cancer therapeutic agent in addition to the anti-VEGF antibody.

22. The method of claim 21, wherein the anti-cancer therapeutic agent is anti-VEGF-C antibody.

23. The method of claim 22, wherein the anti-VEGF antibody and the anti-VEGF-C antibody are administered simultaneously.

24. The method of claim 1, wherein the sample obtained from the patient is a tissue sample.

25. The method of claim 1 further comprising determining immunohistochemistry (IHC) score comprising performing IHC assay for said gene, wherein the IHC score is at least 1+.

26. The method of claim 25, wherein the IHC score is at least 2+.

27. The method of claim 25, wherein the IHC score is 3+.

28. The method of claim 1 further comprising determining whether the sample comprises a tumor cell that expresses VEGFR3, wherein presence of VEGFR3 expression indicates that the patient may benefit from anti-cancer therapy in addition to VEGF antagonist.

29. The method of claim 28, wherein the VEGFR3 expression is mRNA expression.

30. The method of claim 29, wherein the presence of VEGFR3 mRNA expression is determined using qRT-PCR or qPCR.

31. The method of claim 28, wherein the VEGFR3 expression is protein expression.

32. The method of claim 28, wherein the presence of VEGFR3 protein expression is determined using IHC assay.

33. The method of claim 1 further comprising measuring expression level of VEGFR3, wherein the expression level of VEGFR3 in the sample is increased as compared to the reference sample.

34. The method of claim 33, wherein the expression level of VEGFR3 is mRNA expression level.

35. The method of claim 34, wherein the increased mRNA expression level of VEGFR3 is in tumor cells.

36. The method of claim 1 further comprising determining immunohistochemistry (IHC) score comprising performing IHC assay for said gene and determining whether the sample comprises a tumor cell that expresses VEGFR3, wherein the IHC score of at least 1+ and presence of VEGFR3 expression indicates that the patient may benefit from anti-cancer therapy in addition to VEGF antagonist.

37. The method of claim 36, wherein the IHC score is at least 2+.

38. The method of claim 36, wherein the IHC score is 3+.

39. The method of claim 36, wherein the VEGFR3 expression is mRNA expression.

40. The method of claim 39, wherein the presence of VEGFR3 mRNA expression is determined using qRT-PCR or qPCR.

41. The method of claim 36, wherein the VEGFR3 expression is protein expression.

42. The method of claim 41, wherein the presence of VEGFR3 protein expression is determined using IHC assay.

43. The method of claim 1 further comprising determining whether the sample comprises a tumor cell that expresses VEGF-D, wherein presence of VEGF-D expression indicates that the patient may benefit from anti-cancer therapy in addition to VEGF antagonist.

44. The method of claim 43, wherein the VEGF-D expression is mRNA expression.

45. The method of claim 44, wherein the presence of VEGF-D mRNA expression is determined using qRT-PCR or qPCR.

46. The method of claim 43, wherein the VEGF-D expression is protein expression.

47. The method of claim 43, wherein the presence of VEGF-D protein expression is determined using IHC assay.

48. A kit comprising an array comprising polynucleotides capable of specifically hybridizing to one or more genes and to VEGF-A, wherein the kit further comprises instructions for using said array to determine ratios between the expression levels of one of more said genes and VEGF-A to predict responsiveness of a patient to anti-angiogenic therapy or anti-cancer therapy, wherein a change in the ratio between the expression level of at least one of said genes and expression level of VEGF-A in the sample as compared to the ratio between the expression level of the same gene and the expression level of VEGF-A in a reference sample indicates that the patient may benefit from anti-angiogenic therapy or anti-cancer therapy in addition to VEGF antagonist.

49. The kit of claim 48, wherein the change in the ratio is increased.

50. The kit of claim 48, wherein the change in the ratio is decreased.

51. The kit of claim 48, wherein at least one of the said genes is an angiogenic factor.

52. The kit of claim 51, wherein said angiogenic factor is VEGF-C.

53. The kit of claim 51, wherein said angiogenic factor is VEGF-D.

54. The kit of claim 51, wherein said angiogenic factor is bFGF.

55. The kit of claim 51, wherein said angiogenic factor is VEGFR3.

56. A set of compounds capable of detecting expression levels of one or more genes and expression level of VEGF-A to determine ratios between expression levels of one or more genes and expression level of VEGF-A in a sample obtained from a patient, wherein a change in the ratio between the expression level of at least one of said genes and the expression level of VEGF-A in the sample as compared to the ratio between the expression level of the same gene and the expression level of VEGF-A in a reference sample indicates that the patient may benefit from anti-angiogenic therapy or anti-cancer therapy in addition to VEGF antagonist.

57. The set of compounds of claim 56, wherein the compounds are polynucleotides.

58. The set of compounds of claim 56, wherein the compounds are proteins.

59. The set of compounds of claim 58, wherein the proteins are antibodies.

60. The set of compounds of claim 56, wherein at least one of the said genes is an angiogenic factor.

61. The set of compounds of claim 60, wherein said angiogenic factor is VEGF-C.

62. The set of compounds of claim 60, wherein said angiogenic factor is VEGF-D.

63. The set of compounds of claim 60, wherein said angiogenic factor is bFGF.

64. The set of compounds of claim 60, wherein said angiogenic factor is VEGFR3.


SEQUENCE LISTING (21 sequences)

SEQ ID NO: 1 - DNA, 21 bp, Artificial sequence
  sequence is synthesized
agtccctgccctttgtacaca

SEQ ID NO: 2 - DNA, 19 bp, Artificial sequence
  sequence is synthesized
ccgagggcctcactaaacc

SEQ ID NO: 3 - DNA, 23 bp, Artificial sequence
  sequence is synthesized
cgcccgtcgctactaccgattgg

SEQ ID NO: 4 - DNA, 20 bp, Artificial sequence
  sequence is synthesized
caccgtttggctcgatatta

SEQ ID NO: 5 - DNA, 21 bp, Artificial sequence
  sequence is synthesized
ggcagaggctgtagatgattc

SEQ ID NO: 6 - DNA, 21 bp, Artificial sequence
  sequence is synthesized
accaagcgagtcctccctccc

SEQ ID NO: 7 - DNA, 14 bp, Artificial sequence
  sequence is synthesized
accccgacggccga

SEQ ID NO: 8 - DNA, 25 bp, Artificial sequence
  sequence is synthesized
tcttctgcttgaagttgtagcttga

SEQ ID NO: 9 - DNA, 23 bp, Artificial sequence
  sequence is synthesized
tccgggagaagagcgaccctcac

SEQ ID NO: 10 - DNA, 20 bp, Artificial sequence
  sequence is synthesized
gcagaatcatcacgaagtgg

SEQ ID NO: 11 - DNA, 20 bp, Artificial sequence
  sequence is synthesized
tctcgattggatggcagtag

SEQ ID NO: 12 - DNA, 27 bp, Artificial sequence
  sequence is synthesized
tgcgctgatagacatccatgaacttca

SEQ ID NO: 13 - DNA, 20 bp, Artificial sequence
  sequence is synthesized
cagtgtcaggcagcgaacaa

SEQ ID NO: 14 - DNA, 20 bp, Artificial sequence
  sequence is synthesized
cttcctgagccaggcatctg

SEQ ID NO: 15 - DNA, 30 bp, Artificial sequence
  sequence is synthesized
ctgccccaccaattacatgtggaataatca

SEQ ID NO: 16 - DNA, 20 bp, Artificial sequence
  sequence is synthesized
ctgccagaagcacaagctat

SEQ ID NO: 17 - DNA, 21 bp, Artificial sequence
  sequence is synthesized
acatggtctggtatgaaaggg

SEQ ID NO: 18 - DNA, 22 bp, Artificial sequence
  sequence is synthesized
cacccagacacctgcagctgtg

SEQ ID NO: 19 - DNA, 24 bp, Artificial sequence
  sequence is synthesized
acagacagtgggatggtgctggcc

SEQ ID NO: 20 - DNA, 21 bp, Artificial sequence
  sequence is synthesized
caaaggctctgtggacaacca

SEQ ID NO: 21 - DNA, 24 bp, Artificial sequence
  sequence is synthesized
tctctatctgctcaaactcctccg